(19) United States
(12) Patent Application Publication
(10) Pub. No.: US 2018/0200494 A1
(43) Pub. Date: Jul. 19, 2018

(54) DEVICES, SYSTEMS, AND METHODS FOR DELIVERING THERAPEUTIC SUBSTANCES IN A MAMMALIAN BODY

(71) Applicant: BioInspire Technologies, Inc., Palo Alto, CA (US)

(72) Inventors: Simon GATRALL, San Francisco, CA (US); Celia L. CORRENTE, San Francisco, CA (US); Jonathan P. DOWNING, Belmont, CA (US); Arthur E. FERDINAND, San Jose, CA (US)

(73) Assignee: BioInspire Technologies, Inc., Palo Alto, CA (US)

(21) Appl. No.: 15/873,829

(22) Filed: Jan. 17, 2018

Related U.S. Application Data
(60) Provisional application No. 62/447,829, filed on Jan. 18, 2017.

(51) Int. Cl. A61M 31/00

(52) U.S. Cl. CPC A61M 31/007; A61M 2210/0681; A61M 2210/125; A61M 2210/1003; A61M 2210/1035

(57) ABSTRACT

Systems and methods for the delivery of devices such as films to target locations in mammalian bodies are provided herein. The films may be configured to release a therapeutic agent over time, among other features. Delivery devices and methods described herein may be particularly well-suited for the delivery of therapeutic films to a target location in the sinuses.

### CROSS-REFERENCE TO RELATED APPLICATIONS

This application claims the benefit of priority of U.S. Provisional Application Ser. No. 62/447,829, filed Jan. 18, 2017, the entire contents of which is incorporated herein by reference.

### TECHNICAL FIELD

The present technology relates generally to devices and methods for the delivery and placement of medical materials, including biodegradable devices for the delivery of therapeutic substances, within mammalian bodies.

### BACKGROUND

Wounds to the human anatomy may result from interventional, minimally-invasive and/or intraoperative surgical procedures, acts, diseases, and/or underlying conditions. For example, iatrogenic wounds generally are formed during surgery for treating sinusitis, and due to the complicated topology of the sinuses, may take extended periods to heal. In addition, certain portions of the human anatomy are prone to the development of post-operative lesions, which often require treatment via subsequent surgical intervention.

Sinusitis is inflammation of the paranasal sinuses generally due to infection, allergies, or autoimmune issues. Chronic sinusitis affects persons of all age groups and is one of the more prevalent chronic diseases in the United States, affecting 37 million Americans. Chronic sinusitis is generally understood to be sinusitis that persists for 12 weeks or longer. Surgery, although minimally invasive, is generally reserved for acute/intermittent rhinosinusitis and chronic/persistent rhinosinusitis unresponsive to conservative medical treatment or where there are complications associated with those conditions. Functional endoscopic sinus surgery (FESS) of the diseased sinus mucosa is performed to enable ventilation through the natural ostia and restore mucociliary clearance using a minimally invasive endoscopic technique. Although FESS has proven to be an effective procedure in treating chronic sinusitis, the outcome of the surgery can become significantly complicated by operative pathologies, including delayed wound healing, stenosis of the sinus passageways (in 20-30% of cases), adhesions, and the formation of polyps. Various mechanical means such as nasal stents and packings have been developed to aid in postoperative wound care; however, experience has shown that these methods do not provide an effective way of addressing the complications.

Pharmaceutical treatment of iatrogenic wounds with therapeutic agents such as steroids has been shown to reduce postoperative complications. However, there does not exist in the prior art an effective manner for delivering appropriate dosages of therapeutic agents over a desired timeframe within the sinus cavities.

Balloon sinus dilation is a relatively new technique for treating chronic sinusitis by opening blocked passages with balloon inflation. While more limited in application than FESS, this modality may become the treatment of choice for limited or moderate sinus disease. Accordingly, it would be desirable to provide an effective device for delivering a therapeutic agent to sinus tissue over a period of time following balloon sinus dilation.

U.S. Pat. Nos. 8,529,941, 8,563,510, 9,072,681, 9,192,698, and 9,271,925 to Hakimimehr et al., assigned to the assignee of the present invention, the entire contents of each of which are hereby incorporated by reference, describe, inter alia, methods and devices having a free-standing film of solid fibrinogen, and optionally solid thrombin, configured in the form of a thin sheet in various configurations, including multi-layer configurations. These and other devices disclosed therein may be configured to release a therapeutic agent over time, among other features.

It would be desirable to provide systems and methods for the safe and effective delivery into a mammalian body of devices, including those described in the foregoing Hakimimehr patents, that permit the release of different therapeutic agents over the same temporal profiles and/or different therapeutic agents over different temporal profiles.

### SUMMARY

In view of the foregoing, the present invention is directed to methods and systems for delivering a biocompatible film to tissue of a patient. For example, one or more films may be delivered within one or more bodily cavities using the delivery systems such as nasal and/or sinus cavities of the patient. The delivery system may include a sheath having a distal end, a proximal end, and a lumen extending therebetween. In one embodiment, the distal end of the sheath is sized and shaped for insertion through a nostril of the patient and for advancement to at least one of the nasal cavities and/or the sinus cavity. The system also includes a handle disposed adjacent to the proximal end of the sheath, wherein the handle has an actuator.

In addition, the system includes a shaft coupled to the actuator and disposed within the lumen of the sheath. The shaft includes a groove extending between a proximal end and a distal end of the shaft, and the sheath includes a folded portion sized and shaped to align with the groove of the shaft. The folded portion of the sheath may include a slit that provides strain relief as the shaft is disposed within the lumen of the sheath. The longitudinal axis of the distal end of the shaft may be offset by a predetermined angle to a longitudinal axis of a proximal end of the shaft. Additionally, the shaft may include a channel disposed along an upper portion of the shaft, the channel sized and shaped to receive an actuator post.

The system includes a curved end effector coupled to a distal end of the shaft. The curved end effector has a convex outer surface such that the biocompatible film is disposed thereon during delivery. The curved end effector transitions from a delivery state, wherein the curved end effector is disposed within the lumen of the sheath, to a deployed state, wherein the curved end effector is exposed past the distal end of the sheath to deliver the biocompatible film to tissue at the bodily cavity, e.g., nasal cavity and/or the sinus cavity, responsive to actuation at the actuator. For example, the sheath may slide proximally while the shaft and curved end effector remain in place to expose the film past the distal end of the sheath for deployment at tissue. As another example, the shaft and curved end effector may move distally while the sheath remains in place to expose the film past the distal end of the sheath for deployment at tissue. Further still, the sheath and the curved end effector may slide relative to one another to expose the film past the distal end of the sheath. In some aspects, the curved end effector provides a uniform gap between an inner surface of the sheath and the biocompatible film when the sheath is disposed over the convex outer surface of the curved end effector. The convex outer surface of the curved end effector may have a radius of curvature decreasing axially in a direction from the flared distal surface toward the proximal end. Alternatively, the convex outer surface of the curved end effector has a constant radius of curvature along a longitudinal axis of the curved end effector.

The curved end effector also includes a flared distal surface. The flared distal end may include an atraumatic tip for to minimizing trauma or damage to the tissue during delivery. In addition, the curved end effector includes left and right rails extending from the flared distal surface longitudinally along at least a portion of opposite lateral ends of the curved end effector. The left and right rails facilitate consistent and reliable axial sliding of the shaft within the sheath, and keep fluid out of a vicinity of the biocompatible film to avoid premature wetting thereof. The convex outer surface of the curved end effector may include at least one indented portion extending longitudinally along the convex outer surface. The at least one indented portion has a radius of curvature less than a radius of curvature of the curved end effector, such that a surface of the indented portion is separated a predetermined distance from the biocompatible film when the biocompatible film is disposed on the convex outer surface of the curved end effector.

In accordance with one aspect of the present invention, a method for delivering a biocompatible film to a bodily cavity, e.g., nasal cavity and/or a sinus cavity, of a patient is provided. The method includes inserting a distal end of a sheath through a nostril of the patient, the sheath having a shaft disposed within a lumen of the sheath, the shaft having a curved end effector coupled to a distal end of the shaft and having a convex outer surface for having the biocompatible film disposed thereon during delivery; advancing the distal end of the sheath to the bodily cavity, e.g., nasal and/or sinus cavities; actuating an actuator disposed adjacent a proximal end of the sheath to transition the curved end effector from a delivery state, wherein the curved end effector is disposed within the lumen of the sheath, to a deployed state, wherein the curved end effector is exposed past the distal end of the sheath; and delivering the biocompatible film to tissue at the cavity. The method may be repeated to deliver additional films in the same cavity or in other bodily cavities.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure and are not intended to limit the scope of the present technology.

FIGS. 1A-1B are perspective views of a film delivery system configured in accordance with an embodiment of the present technology.

FIG. 2 is an exploded view of a film delivery system configured in accordance with an embodiment of the present technology.

FIG. 3 is a perspective view of a handle first half of a film delivery system configured in accordance with an embodiment of the present technology.

FIG. 4 is a perspective view of an actuator or thumb slider of a film delivery system configured in accordance with an embodiment of the present technology.

FIG. 5A is a perspective view of a shaft of a film delivery system configured in accordance with an embodiment of the present technology.

FIG. 5B is an elevation of the shaft of FIG. 5A.

FIG. 5C is a perspective cutaway cross section of the shaft of FIG. 5A taken along lines 5C-5C in FIG. 5B.

FIG. 5D is a top view of the shaft of FIG. 5A.

FIG. 5E is a partial elevation of the shaft of FIG. 5A detailing an offset between the shaft central angled portion and the shaft proximal portion.

FIGS. 6A-6C are various views of the distal portion of the shaft of FIG. 5A.

FIG. 6D is a perspective view of the shaft distal portion of FIG. 6C schematically detailing a film and portion of a sheath thereover.

FIG. 6E is a partial perspective exploded view of a distal portion of a shaft of a film delivery system configured in accordance with another embodiment of the present technology schematically detailing a film thereover.

FIGS. 6F-6G are partial perspective end top and bottom views, respectively, of the shaft distal portion of FIG. 6C detailing a configuration of film and sheath layers thereover and the shaft flared end geometry.

FIG. 6H is a partial perspective view of a distal portion of a shaft of a film delivery system configured in accordance with a further embodiment of the present technology.

FIGS. 6I-6J are a perspective view of a distal portion of a shaft of a film delivery system configured in accordance with further embodiments of the present technology.

FIGS. 7A-7C are various views of a sheath of film delivery system configured in accordance with an embodiment of the present technology.

FIG. 8 is a schematic plan view of a film configured for use in a film delivery system configured in accordance with an embodiment of the present technology.

FIG. 9 is a perspective phantom assembly view of a film delivery system configured in accordance with an embodiment of the present technology.

FIG. 10 is a partial cross-section elevation of the film delivery system of FIG. 9.

FIG. 11A is a perspective partial cutaway view of a handle first half and actuator or thumb slider of a film delivery system configured in accordance with an embodiment of the present technology.

FIGS. 11B-11C are partial cutaway elevations of the handle first half and actuator or thumb slider of FIG. 11A showing two different axial positions of actuator or thumb slider within the handle first half.

FIG. 12A is an elevation of the handle first half of FIG. 3.

FIG. 12B is an elevation of the handle first half of FIG. 3 with the shaft of FIG. 5A.

FIGS. 13A-13D are partial perspective views of the shaft of FIG. 5A, actuator or thumb slider of FIG. 4 and sheath of FIGS. 7A-7C.

FIGS. 14A-14D are partial cross section elevations of the film delivery system of FIG. 1 detailing proximal axial motion of the actuator or thumb slider and the sheath.

FIGS. 15A-15G illustrate a method to deploy a film into the ethmoid sinus using a film delivery system configured in accordance with an embodiment of the present technology.

FIGS. 16A-16B are elevations of a distal portion of a modified forceps film delivery system configured in accordance with another embodiment of the present technology.

FIG. 17 is a perspective view of a distal portion of a sponge tip film delivery system configured in accordance with yet another embodiment of the present technology.

FIGS. 18 and 19A-19C are perspective views of a wireform release delivery system configured in accordance with yet another embodiment of the present technology.

FIGS. 20A-20C are perspective views of a distal portion of a rotating tine film delivery system configured in accordance with yet another embodiment of the present technology.

FIGS. 21A-21C are perspective views of a distal portion of a suction tip film delivery system configured in accordance with yet another embodiment of the present technology.

FIGS. 22A-22D are perspective views of a distal portion of a passive applicator film delivery system configured in accordance with yet another embodiment of the present technology.

FIGS. 23A-23F are perspective views of a retracting hooks film delivery system configured in accordance with yet another embodiment of the present technology.

FIGS. 24 and 25A-25F depict a film delivery system in the form of a cartridge tip applicator in accordance with yet another embodiment of the present technology.

FIGS. 26A-26B are perspective views of a lead screw film delivery system configured in accordance with yet another embodiment of the present technology.

FIGS. 27-28 are perspective views of a belt-guided film delivery system configured in accordance with yet another embodiment of the present technology.

FIGS. 29 and 30A-30B are perspective views of a push tube film delivery system configured in accordance with yet another embodiment of the present technology.

### DETAILED DESCRIPTION

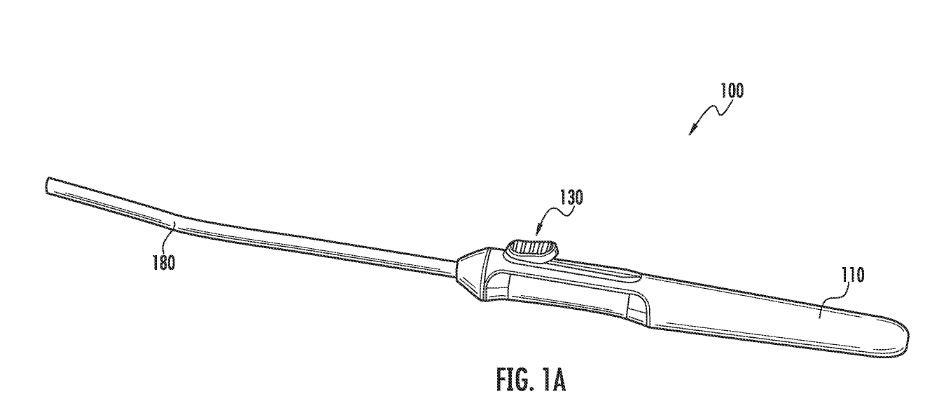

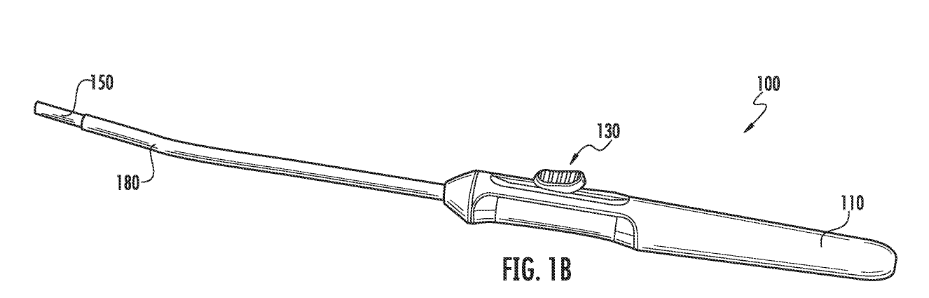

The present technology provides devices, systems, and methods for the delivery of devices such as films to target locations in mammalian bodies that may be configured to release a therapeutic agent over time, among other features. Specific details of several embodiments of delivery devices, systems and associated methods in accordance with the present technology are described below with reference to FIGS. 1A-30B. Although many of the embodiments are described below with respect to delivery devices, systems, and methods for the delivery of devices such as therapeutic films to a target location in the sinuses, other applications and other embodiments in addition to those described herein are within the scope of the technology. For example, one or more film devices may be delivered to one or more hollow body organs, such as the esophagus, stomach, intestines, bronchus, trachea, lungs, urethra, ureters, the sinuses, the ears, eyes, or the heart and optionally exposed to moisture at bodily tissue, e.g., the wall of the organ. In embodiments configured for use in a heart, the device or film may be used to treat or seal a patent foramen ovale, a paravalvular leak, the left auricular appendage, or the like. In other embodiments, the devices or films may be used to modify the geometry of the left ventricle, and thus reduce functional mitral regurgitation. Such films also may be delivered to target locations that include wounds resulting from interventional, minimally-invasive and/or intraoperative surgical procedures, diseases, and/or underlying conditions.

In one proposed application, a delivery device is advanced using a delivery system of the present technology to a target location in the sinuses and is used to deploy a film containing a therapeutic substance to bodily tissue within the sinus to treat iatrogenic wounds resulting from surgical procedures for treating, for example, sinusitis. The delivery system may be used to apply a film to areas such as, but not limited to, a paranasal sinus, ethmoid sinus, ethmoidectomy channel, and frontal sinus outflow tract. A device or film may also be delivered using a delivery system of the present technology to the osteomeatal complex, along the maxillary ostium or any sinus insertion device capable of accessing the maxillary ostium. Such a device or film, including one in a multi-layer configuration, provides many advantages for treating wounds, such as iatrogenic wounds resulting from surgical procedures for treating sinusitis.

In such sinus and other applications, one advantage in using such films is that a therapeutic agent, such as a steroidal anti-inflammatory agent, may be delivered locally with minimal systemic exposure to significantly improve the outcome of surgery. Another advantage is that the device or film may be configured to naturally biodegrade so there is no need for subsequent removal. A further advantage is that the device or film improves the healing of the mucosal lining so as to reduce healing time, and thus, reduce the likelihood of the formation of adhesions. Yet another advantage is that the device may be configured to seal the mucosal wall, which may prevent adhesions of the opposing surfaces in the sinuses. Another advantage is that the device or film may be configured to adhere to the sinus wall of the nasal passageway in a thin layer such that it does not obstruct the flow of air and liquid, thereby improving a patient's quality of life after surgery. Yet a further advantage is that because in such a configuration the device or film adheres to the sinus wall, rather than being retained in position by mechanical force from, for example, a stent, further damage to the mucosal layer is prevented, which in turn leads to improved patient quality of life.

In another application, a device or film is advanced using a delivery system of the present technology to a target location in the bronchus and applied to bodily tissue at an anastomosis site within the bronchus to treat post-operative lesions following a surgical procedure, such as lung transplantation. Bronchial stenosis following lung transplantation is one such problem, often arising from scar stenosis at the bronchial anastomotic site with or without previous anastomotic dehiscence. The device or film may be delivered to the anastomosis site using a delivery system of the present technology and applied to bodily tissue using the adhesive properties of the device. The device or film may release therapeutic agent(s) such as cyclosporine or another antiproliferative agents that promote healing while the device biodegrades. The device or film may be applied to bodily tissue surgically or during an anastomosis procedure.

In a further application, a device or film is advanced to a target location using a delivery system of the present technology in the larynx, trachea, carina, or bronchi and applied to bodily tissue at a stenosed area within the larynx, trachea, carina, or bronchi to treat post-operative lesions resulting from surgical procedures for treating, for example, airway stenosis, such as post-intubation tracheal stenosis. The device or film may be delivered to the affected area using a suitable delivery system, e.g., as described herein alone or in conjunction with balloon dilation opening the restricted passageway. After application, the device or film may release therapeutic agent(s) such as antiproliferative agents and/or anti-inflammatory agents to the affected site to improve the outcome of the balloon dilation.

In yet another application, a device or film is advanced using a delivery system of the present technology to a target location in a neck in a lobe of the lung and applied to bodily tissue at the wall of the lobe to treat wounds from, for example, asthma. The device or film may release therapeutic agent(s) such as steroidal anti-inflammatory agents to the lungs. The device or film is believed to minimize the systemic exposure to steroids and to improve patient compliance with treatment.

In another application, a device or film is advanced using a delivery system of the present technology to a target location in the trachea or bronchi and applied to bodily tissue at an ulcer at the trachea or bronchi to treat lesions resulting from tumors, for example, squamous cell carcinomas. The device or film may be used to aid in the treatment of bronchial tumor resection or weakened bronchia due to external beam radiation therapy. The device or film may release therapeutic agent(s) such as chemotherapeutics (e.g., antiproliferative agents or antibody based therapies), anti-inflammatory agents, and/or antibiotics to the bodily tissue. The device or film may be delivered after open surgery or during an endoscopic procedure such as a bronchoscopy.

In yet another application, a device or film is advanced using a delivery system of the present technology to a target location in the body and applied to bodily tissue at an internal adhesion or (potential) dermal scar to treat lesions resulting from, for example, invasive surgical procedures. The device or film may be used in preventing or reducing the size of internal adhesions or dermal scarring. An adhesion is a band of scar tissue that binds together two internal body surfaces. Adhesions can cause subsequent health issues such as pain (back and abdominal), infertility, and digestive issues resulting in increased costs and potential secondary surgical interventions. The device or film may act as a barrier between two body surfaces during the healing process; it then biodegrades and provides a space between the two surfaces. The device or film also may release therapeutic agent(s) such as anti-inflammatory agents, antiproliferative agents, antibiotics, and/or mitomycin C to promote healing of an adhesion or scar.

In another application, a device or film is advanced using a delivery system of the present technology to a target location in or on the body and applied to bodily tissue to promote healing. The device or film may release therapeutic agent(s) such as antibiotic agents, antimicrobial agents, antifungal agents, growth factors, and/or analgesic agents to promote wound healing. For example, a device or film for treating diabetic ulcers may release an antibiotic agent. Advantageously, since the device or film is biodegradable, it does not need to be removed before a new dressing is put in place and therefore prevents interruption of wound healing due to change of dressing.

In a further application, a device or film is advanced by a delivery system of the present technology to a target location in or on the body and applied to bodily tissue at a site of pain to treat pain and/or inflammation. For example, the device or film may be used to treat radicular pain and sciatica of the lower back or articular pain of the joint. The device or film may be advanced to a target location at a joint or a space inside the body using a trocar delivery device. In one embodiment, the device or film is inserted minimally invasively via epidural trocar into the foraminal or interlaminar space of the lumbar spine. The device or film may be configured to release therapeutic agent(s) such as anti-inflammatory agents, analgesic agents, anti-infective agents, and/or anti-proliferative agents. The device or film may contain multiple film-layers for the programmed release of different therapeutic agents with different temporal profiles. For example, acute analgesics such as the—caine derivatives may be delivered immediately and over a duration of up to the first 8-12 hours, while an anti-inflammatory agent may be delivered for a much longer period. In another application, the device or film may be applied by a delivery system of the present technology after an open or minimally invasive surgery (laparoscopic surgery) to deliver therapeutic agents to improve the outcome of the surgery. For example, a device or film with a steroidal anti-inflammatory agent may be applied after a laparoscopic spinal surgery to reduce inflammation and pain after surgery. In addition, therapeutic agent(s) for promoting wound healing may alleviate the side effects of epidural steroid injections such as dural puncture and prevent cerebral spinal fluid leakage.

In yet another application, a device or film may be advanced by a delivery system of the present technology to a target location in the eye and applied to bodily tissue within or on the eye. In one embodiment, the device or film may be applied to the posterior segment of the eye via the vitreous or suprachoroidal space using a cannula, e.g., a 20 to 25 gauge cannula. The device or film also may be applied after vitreoretinal surgery to release a therapeutic agent(s) such as an anti-inflammatory agent over a period of time. The device or film may biodegrade slowly over time thus obviating the need for subsequent surgical extraction and may deliver a sustained profile of therapeutic agent. The adhesive properties of the device or film allow it to be applied in contact with and adhering to the macular surface within the eye so as to avoid visual problems associated with an untethered device in the vitreous.

In another application, a device or film is advanced using a delivery system of the present technology to a target location that is a tumor anywhere in or on the body and applied to bodily tissue at the tumor as a primary mode of treatment or in conjunction with surgery or a minimally invasive surgery as a maintenance therapy to prevent tumor re-growth. The device may release therapeutic agent(s) such as chemotherapeutics including anti-proliferative agents to the site of the tumor. Examples for this application include delivering the device or film having an anti-cancer therapeutic agent to a tumor of the bladder; delivering a device or film having an anti-cancer therapeutic agent to a tumor of the airway, and delivering the device or film having an anti-cancer agent to a resected tumor of the pancreas.

In yet another application, a device or film is advanced using a delivery system of the present technology to a target location in the heart or in a blood vessel and applied to bodily tissue at a wall of the heart or vessel to treat wounds from, for example, cardiac disease or surgical procedures for treating cardiac disease.

Additionally, the device or film may promote endothelialization by delivery of growth factors. In still other instances, one or more devices or films may be used to attach endothelial cells to the inside of the lumen to promote healing. In yet another proposed application, a device or film may be delivered using a delivery system of the present technology within a patient's bronchus or trachea to deliver chemotherapy or an anti-cancer drug through the patient's vasculature or tissue.

As described in U.S. Pat. Nos. 8,529,941, 8,563,510, 9,072,681, 9,192,698, 9,271,925, and 9,446,103, embodiments of the device or film for delivery to a target location may have a variety of configurations, geometries, and dimensions, depending on the specific use for which it is designed. For instance, the device may be a free-standing film of solid fibrinogen, and optionally solid thrombin, configured in the form of a thin sheet in various configurations, including single- and multi-layer configurations. For purposes of the present description, the term “film” may be used to describe not only single- and multi-layer film-based devices but also refers to any construction or configuration of the device that is intended to be delivered to a target location by a delivery system, embodiments of which are described below.

Additionally, several other embodiments of the technology can have different states, components, or procedures than those described herein. Moreover, it will be appreciated that specific elements, substructures, advantages, uses, and/or other features of the embodiments described with reference to FIGS. 1A-30B can be suitably interchanged, substituted or otherwise configured with one another in accordance with additional embodiments of the present technology. For example, alternative designs for sheath 180 and distal portion 156 of shaft 150 may be used with the handle 110 and actuator 130 as described herein. Furthermore, alternative end effector designs as described herein in the various Figures; namely, various configurations for deploying a film to a target tissue location, may be used with the various delivery system 100 components as described herein or with modification as necessary. A person of ordinary skill in the art, therefore, will accordingly understand that the technology can have other embodiments with additional elements, or the technology can have other embodiments without several of the features shown and described below with reference to FIGS. 1A-30B.

With regard to the terms “distal” and “proximal” within this description, unless otherwise specified, the terms can reference a relative position or location of the portions of the therapeutic devices described herein as well as the various delivery device embodiments. In addition, such terms may be used with reference to an operator and/or a location in a mammalian lumen, such as, e.g., those for accessing the sinus cavities.

I. Selected Film Delivery Systems and Associated Methods

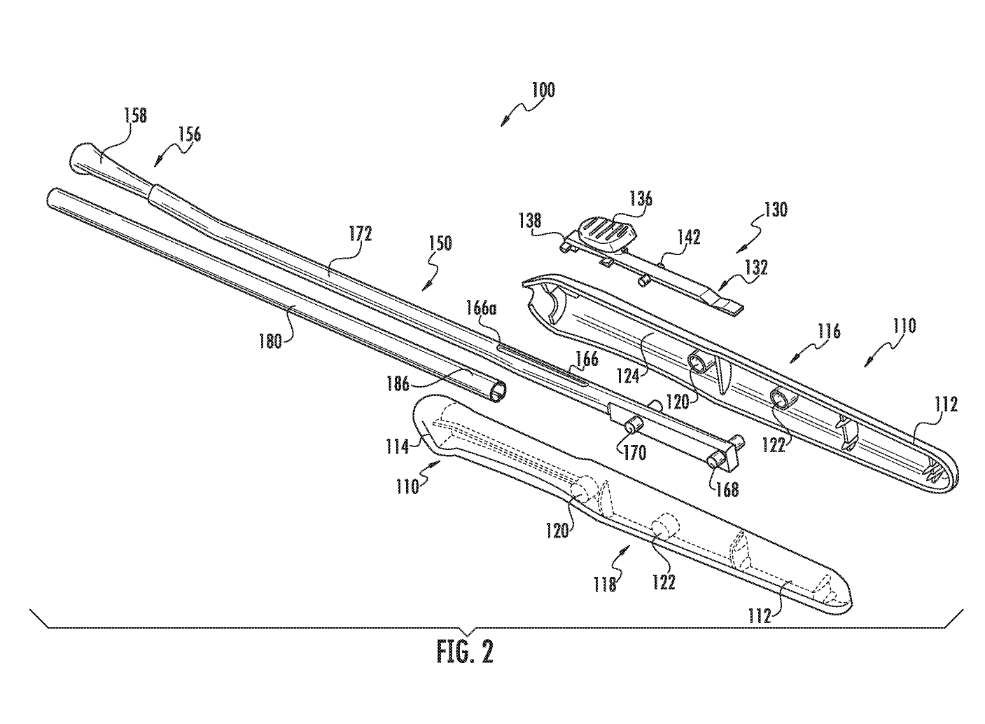

Referring to FIGS. 1A-15G, embodiments of film delivery system 100 in accordance with the present technology is depicted. Film delivery system 100 may be used to deliver one or more therapeutic films to a target mammalian tissue. The embodiments of FIGS. 1A-15 are suited to deliver one or more films to, e.g., structures of the sinus, including the ethmoid sinus. As shown with respect to FIGS. 1A-2, system 100 can include handle 110, actuator 130, shaft 150 and sheath 180. Distal portion 156 of shaft 150 may be configured to be delivered to a treatment site proximate the wall of a body lumen (e.g., in the case of a sinus indication, the sinus wall) and positioned so that the shaft distal tip or curved end effector 158 carrying a film (not shown in FIGS. 1A-2) is adjacent the wall at the treatment site so that the film may be delivered to the target tissue. In embodiments of the present technology that are suited for other indications, such as for the deployment of one or more films in the vasculature, shaft 150 can be, e.g., configured to be slidably received within a lumen of a delivery and/or guide catheter (not shown) for intravascular delivery to the treatment site. FIG. 1A depicts system 100 with sheath 180 completely covering shaft 150, in a configuration contemplated prior to deployment of a film as will be described below in greater detail. System 100 includes a curved end effector coupled to the distal end of shaft 150. The curved end effector may be formed as a common piece with shaft 150 or they may be separate pieces. In the delivery state illustrated in FIG. 1A, the curved end effector is disposed within the lumen of sheath 180. FIG. 1B depicts system 100 in a deployed state, wherein the curved end effector is exposed past the distal end of sheath 180 to deliver the biocompatible film to tissue at the bodily cavity e.g., nasal cavity or the sinus cavity, responsive to actuation at actuator 130. Illustratively, the curved end effector has a convex outer surface configured to have the biocompatible film disposed thereon during delivery and during deployment. The convex outer surface preferably faces the tissue where the biocompatible film will be deployed during deployment for ease of release of the film from the convex outer surface upon the biocompatible film coupling to the tissue. FIG. 1B illustrates sheath 180 partially retracted via proximal motion of actuator 130, exposing shaft 150, in preparation for the deployment of a film (not shown) as described in detail below.

System 100 includes handle 110 having proximal portion 112 and distal portion 114. Handle 110 is of a two-piece construction with handle first half 116 and handle second half 118. Other constructions, including unitary and other multiple piece configurations, are possible. For brevity, the following description of the features of handle 110 are provided in terms of handle first half 116 (FIGS. 3, 12A-12B, 14A-14D), although in embodiments identical or similar features can be located on the handle second half 118.

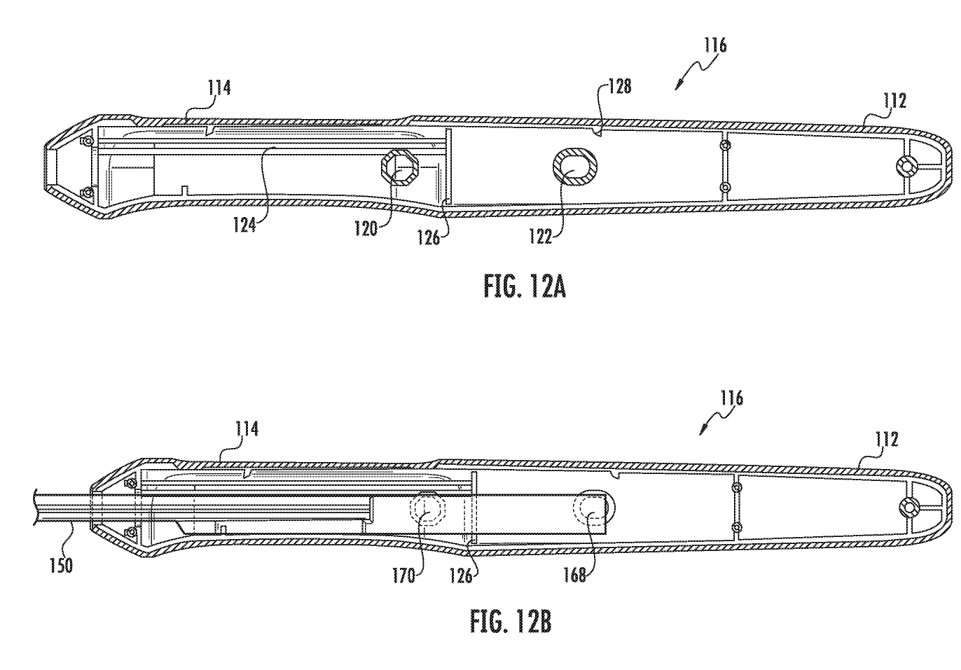

As will be described in greater detail below in connection with, e.g., FIGS. 12A and 12B, distal peg receiving shaft aperture 120 and proximal peg receiving shaft aperture 122 of handle 110 each receives peg 170, 168, respectively, of shaft 150. Shelf 124 of handle 110 is configured to receive and provide a linear pathway for motion of distal boss 138 and proximal boss 142 of actuator 130, and stop 126 is configured to engage with proximal boss 142 of actuator 130. Handle 110 may be made of any suitable material amendable to sterilization, ease of manufacture, ergonomic, cost and other performance factors. Handle first half 116 and second half 118 may be bonded during assembly by any suitable technique as known by those of skill in the art for medical devices, including ultrasonic welding, the use of adhesives, etc.

Turning now to FIG. 4, actuator or slider 130 is shown in perspective view. Actuator 130 the component activated by a user such as a physician to safely retract sheath 180 and expose one or more films 200 for deployment at the target body location. Actuator or slider 130 includes proximal portion 132, distal portion 134, finger or thumb pad 136, distal boss 138 and post or extension 140, proximal boss 142, and locking tooth 144 to facilitate the single use of delivery system 100 as well as other advantages as described below. Actuator 130 may be made of any suitable material that is amendable to sterilization, that has the appropriate mechanical properties and that ranks favorably in other categories including cost, biocompatibility and manufacturability. Candidate materials include but are not limited to, e.g., PETG, polycarbonate and combinations thereof.

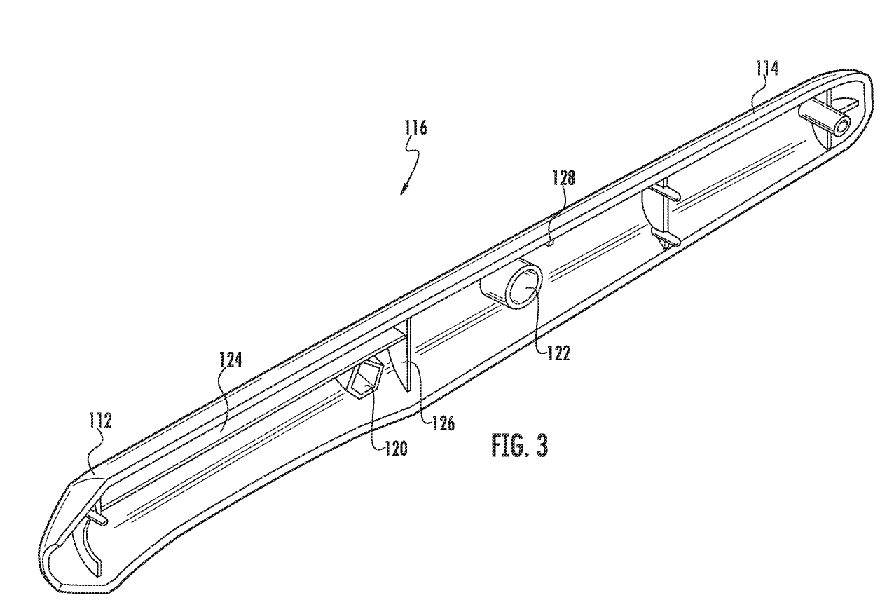

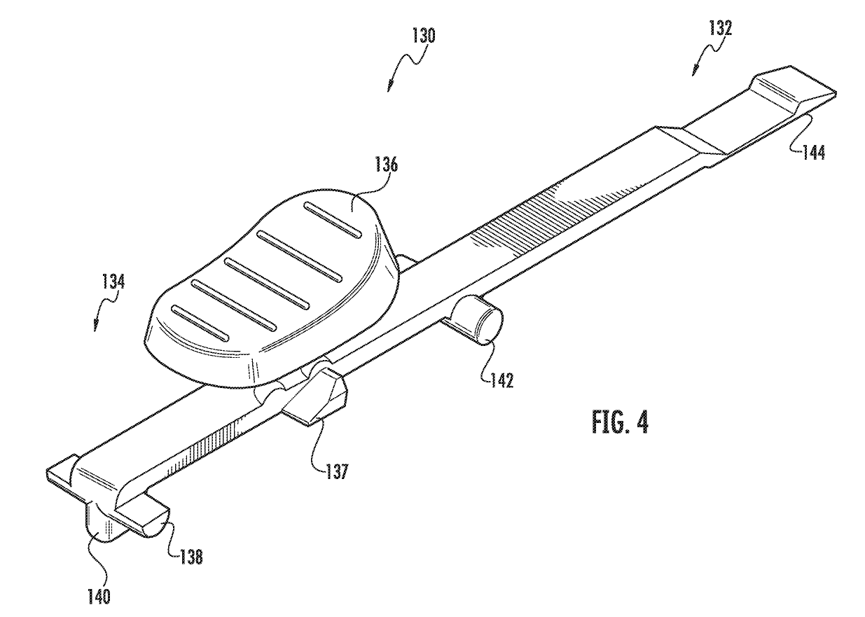

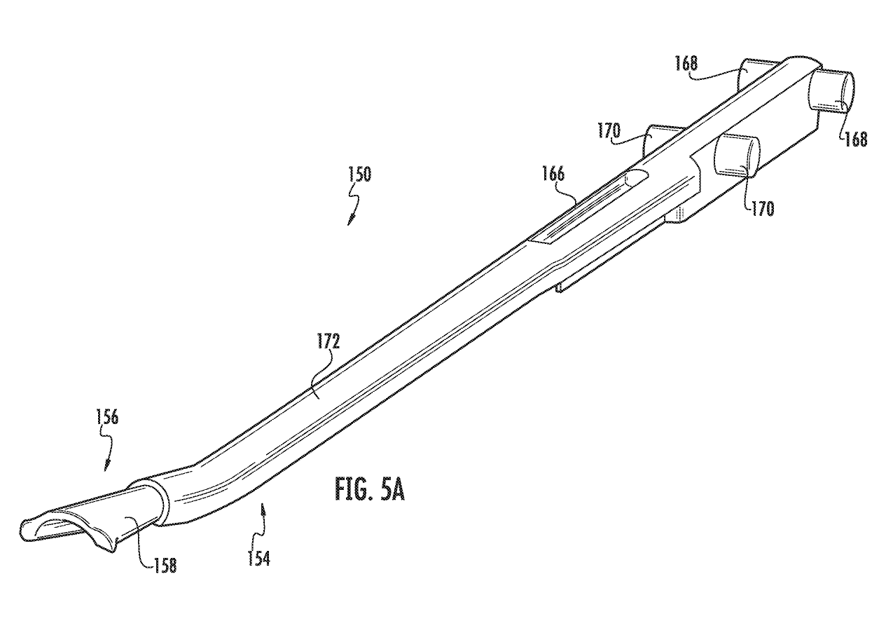

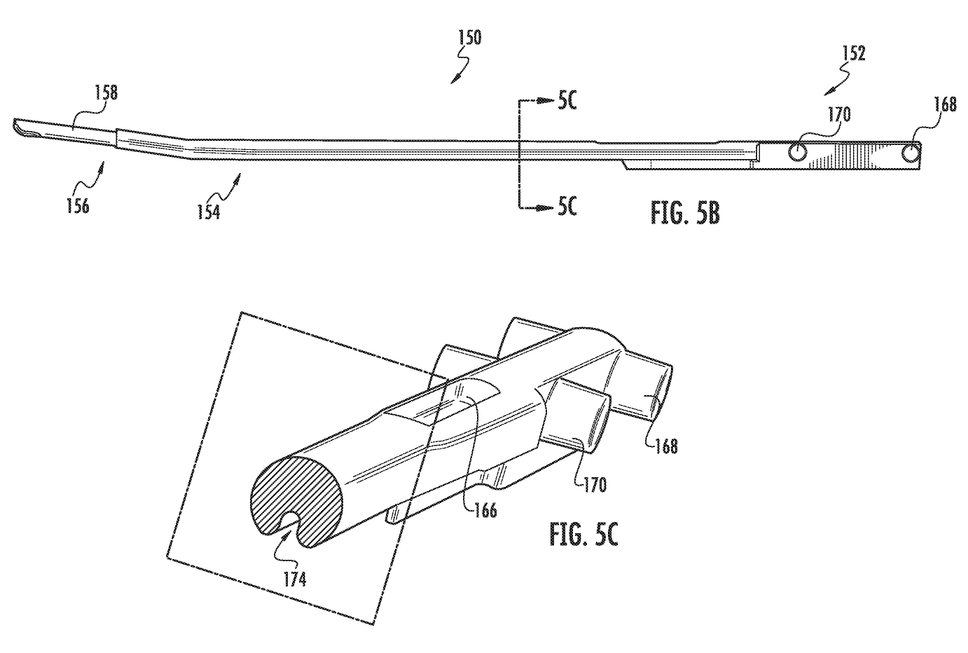

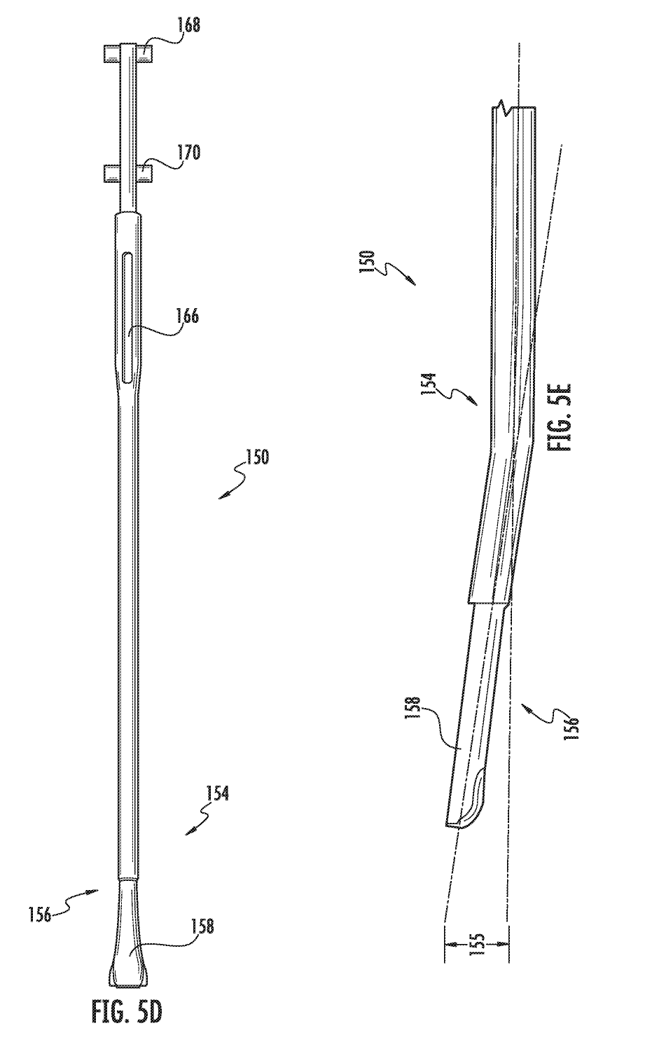

Shaft 150 as shown in FIGS. 5A-5D includes proximal portion 152 with proximal peg 168 and distal peg 170, central angled portion 154, and distal portion 156 terminating in a distal tip or curved end effector 158. Channel 166 is disposed along an upper portion of shaft 150 for receiving actuator post or extension 140. FIG. 5C details in perspective cross-section a groove 174 disposed along the lower length of shaft 150 for receiving sheath 180.

As seen in FIG. 5E, a longitudinal axis of a distal end of shaft 150, e.g., distal portion 156, is offset by a predetermined angle to a longitudinal axis of a proximal end of shaft 150, e.g., proximal portion 152. For example, central angled portion 154 provides offset 155 to distal portion 156 of shaft 150 to facilitate film deployment. The length of offset 155 as measured at the distal end of shaft distal tip 158 may be between about 1 and about 20 mm or greater, between about 4 and 10 mm, between about 5 and about 8 mm, or other lengths as necessary for the indication for which delivery system 100 is designed. A useful offset length for sinus applications is between about 7 and about 8 mm. Such offsets allow the physician to navigate the delicate bony tissues to access a target tissue site while minimizing the potential for inadvertent trauma to the tissue that might otherwise be present without such an offset.

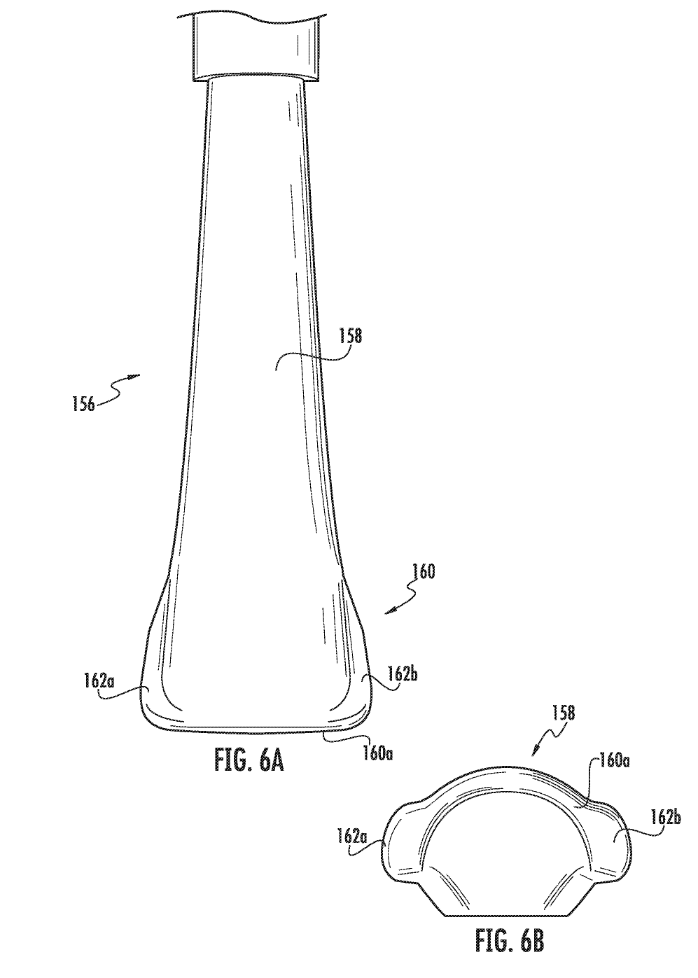

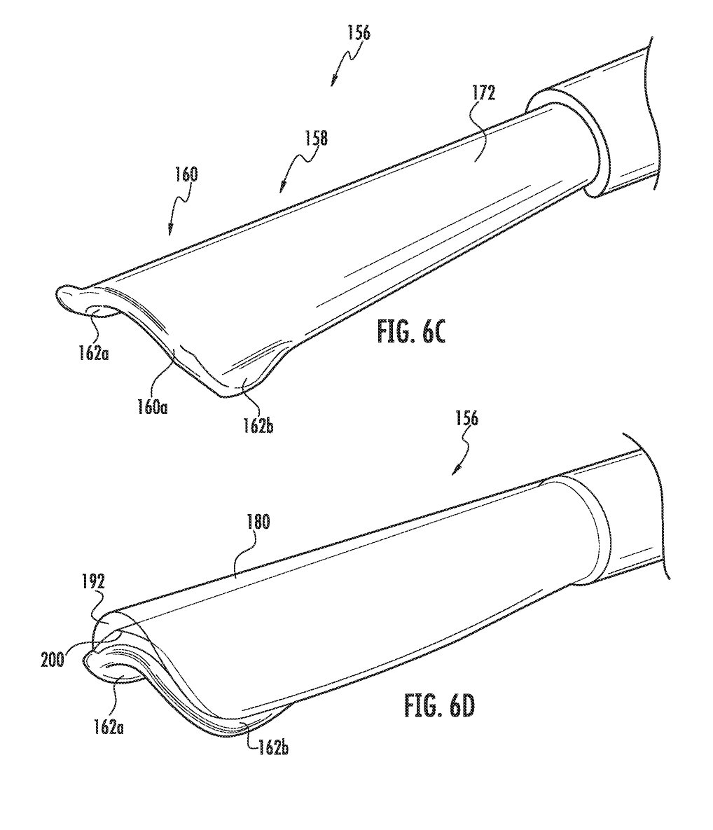

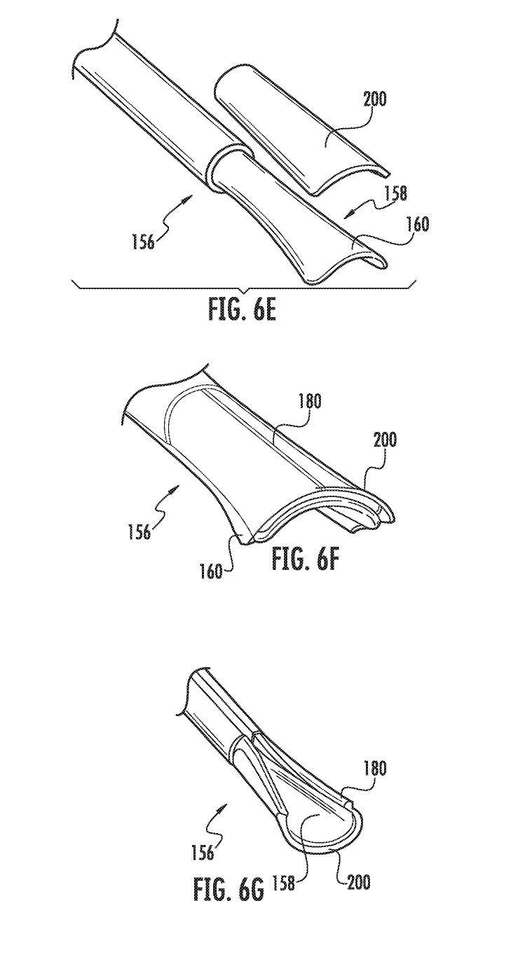

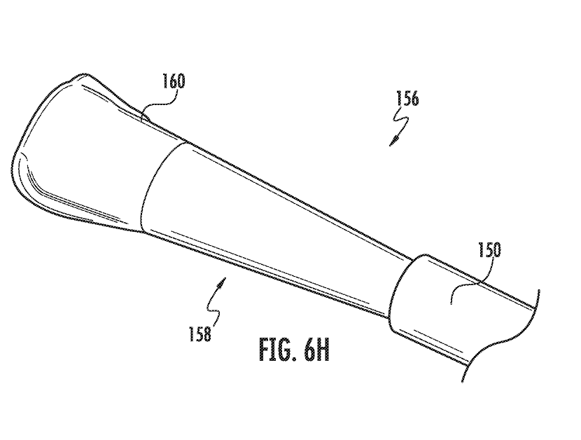

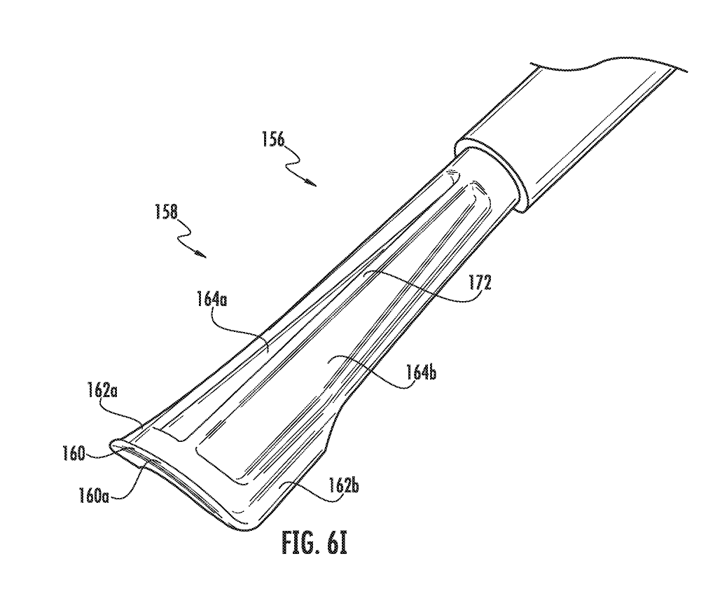

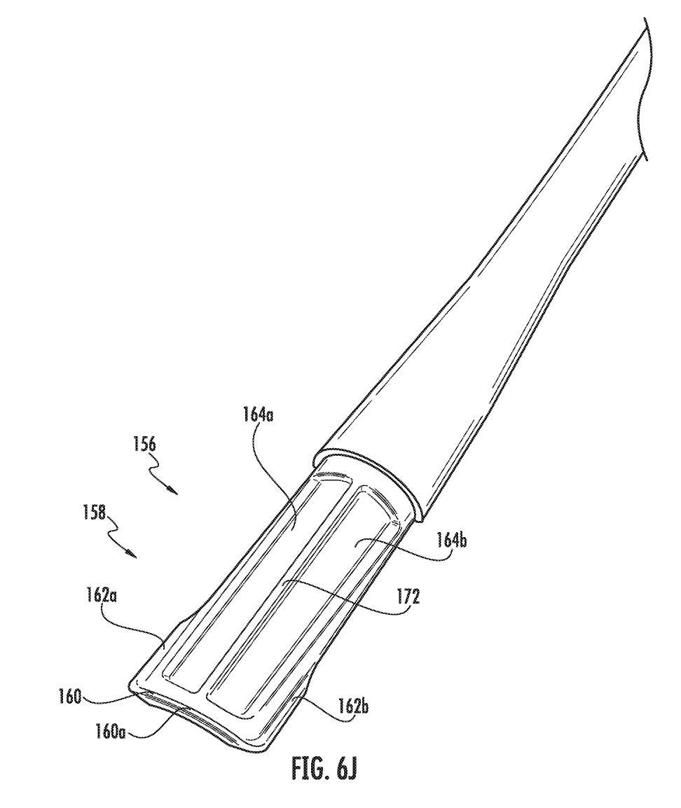

Embodiments of shaft distal portion 156, including in particular distal tip or curved end effector 158, are discussed below in greater detail with reference to FIGS. 6A-6H. Curved end effector 158 includes flared end 160 having distal surface 160a, and left and right rails, e.g., rails 162a, 162b. The left and right rails extend from flared distal surface 160a longitudinally along at least a portion of opposite lateral ends of curved end effector 158. In FIG. 6D, film 200 is schematically shown disposed between shaft outer surface 172 at distal tip 158 and sheath 180 inner surface 192 at sheath distal portion 184. Illustratively, outer surface 172 has a convex shape that preferably faces the wall of the bodily cavity during deployment to facilitate film delivery to tissue within the bodily cavity that may have curved surfaces. A distal surface 160a of flared end 160 may be rounded or otherwise shaped to provide an atraumatic tip to shaft 150. This feature minimizes the potential for unwanted trauma or damage to tissue during operation. For example, shaft flared end distal surface 160a may be formed to have a radius of curvature of, e.g., from about 0.0200 mm to about 0.150 mm, from about 0.0400 mm to about 0.100 mm, from about 0.500 mm to about 0.800 mm, or about 0.075 mm. Other embodiments of shaft distal tip 158 are possible within the scope of the present technology; in particular, those having geometric or design variations at shaft flared end 160. Examples of such variations include those that have differing degrees of flare relative to the radial dimensions of shaft 150 and the dimensions of film 200 and sheath 180, embodiments without side rails 162a and 162b, embodiments using different materials for shaft distal tip 158, embodiments with differing radii of curvature of flared end distal surface 160a, and embodiments having differing degrees of flared end 160 curvature when viewed in axial cross sectional and/or end views. For example, FIG. 6E shows an embodiment of film 200 in relation to an embodiment of distal tip 158 that does not have any side rails.

For example, the curvature, or shape of flared end 160 may vary along the length of distal tip 158 and may take on any number of configurations, such as a circular radius of curvature (e.g., as seen in FIG. 6B's depiction of the distalmost end of shaft 150 in an axial elevation view). This shape generally may be constant or it may vary along the length of distal tip 158. For instance, in the embodiments of FIGS. 6A-6G, a radius of curvature of flared end 160 decreases axially as one moves in a proximal from the flared end distal surface 160a in direction towards shaft central portion 154. It may also take on shapes other than a circular radius of curvature. The shape of this flared end 160 helps to minimize the tendency for sheath 180 to pinch film 200 and promotes the creation and maintenance of a uniform gap between sheath inner surface 192 and film 200 as they are disposed over shaft outer surface 172 in the vicinity of shaft distal tip 158. Shaft flared end 160 also provides a larger surface area to match the appropriately-sized film 200 onto which it is assembled.

FIG. 6H depicts an alternative embodiment for shaft distal portion 156, including shaft distal tip or curved end effector 158 and shaft flared end 160. In this embodiment, shaft flared end 160 includes an elastomeric tip of a different construction that that of the rest of shaft distal tip 158. This configuration provides a relatively soft, non-stick and atraumatic tip that can more flexibly move against the target tissue so to improve the chance of film adherence to the tissue and to promote film release therefrom. It may have unique utility in, e.g., a superior wall of a sinus cavity or other lumen where gravity may play a role in preventing sufficient film adherence to the tissue.

FIGS. 6I and 6J depict alternative embodiments for shaft distal portion 156, including shaft distal tip or curved end effector 158 and shaft flared end 160. Curved end effector 158 includes flared end 160 having distal surface 160a, and left and right rails, e.g., rails 162a, 162b. In this embodiment, curved end effector 158 includes indented portions 164a, 164b extending longitudinally along shaft outer surface 172. Indented portions 164a, 164b have a radius of curvature less than the radius of curvature of shaft outer surface 172 of curved end effector 158. Therefore, when film 200 is disposed on shaft outer surface 172 of curved end effector 158 during delivery, film 200 is separated a predetermined distance from the surface of indented portions 164a, 164b. As will be understood by one of ordinary skill in the art, curved end effector 158 may include more or less than two indented portions on shaft outer surface 172.

The shape of curved end effector 158 generally may be constant or it may vary along the length of distal tip 158. For instance, in the embodiment of FIG. 6I, a radius of curvature of flared end 160 decreases axially as one moves proximally from the flared end distal surface 160a in the direction towards shaft central portion 154. Accordingly, a radius of curvature of indented portions 164a, 164b also decreases axially as one moves proximally from the flared end distal surface 160a in the direction towards shaft central portion 154. It may also take on shapes other than a circular radius of curvature. The shape of this flared end 160 helps to minimize the tendency for sheath 180 to pinch film 200 and promotes the creation and maintenance of a uniform gap between sheath inner surface 192 and film 200 as they are disposed over shaft outer surface 172 and indented portions 164a, 164b in the vicinity of shaft distal tip 158. In the embodiment of FIG. 6J, curved end effector 158 has a constant radius of curvature along its longitudinal axis.

Shaft 150 may be made of any suitable material that is amendable to sterilization, that has the appropriate properties, and that ranks favorably in other categories such as cost, biocompatibility, manufacturability and other performance factors. In addition, shaft distal portion 156, including distal tip or curved end effector 158, may include one or more optional coatings on its outer surface 172 to facilitate reliable release of film 200 during deployment. Coatings such as sodium stearate, oleo- and/or hydrophobic coatings, etc. may be used.

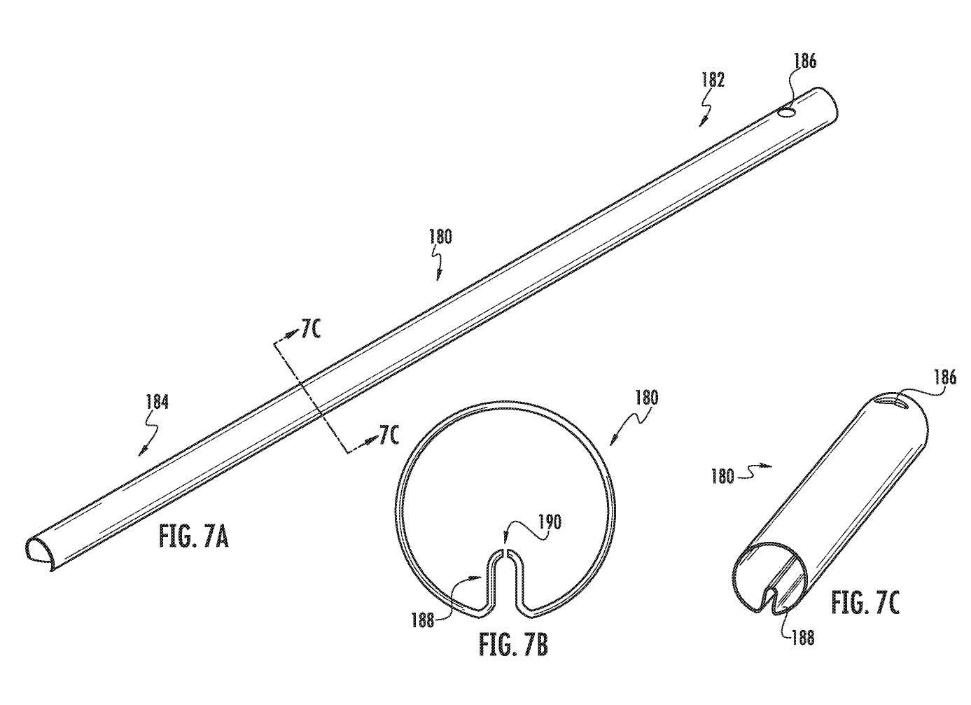

In the embodiment of FIGS. 1A-15G, and as best seen in FIGS. 7A-7C, sheath 180 is shown having proximal portion 182, distal portion 184 and post aperture 186 for receiving actuator post or extension 140. Sheath 180 also includes folded portion 188 and slit 190 along all or a portion of its length. Sheath 180 may be made from any suitable material that is amendable to sterilization, that has the appropriate mechanical properties and that ranks favorably in other categories including cost, manufacturability, chemical resistance, hydrophobicity, stability for gamma radiation/sterilization techniques, dimensional stability, biocompatibility, amenability to extrusions and post-extrusion processing (such as the introduction of slit 190 and compliance for fitting over shaft distal tip or curved end effector 158 and shaft angled portion 154), visual transparency (for ease of visualization during film deployment), etc. Candidate materials for sheath 180 that meet these requirements include polyamides (e.g., aliphatic or semi-aromatic versions such as nylon).

Sheath 180 may be of a unitary construction and may be extruded as a tube of uniform diameter or otherwise manufactured as known by those of skill in the art. During assembly of delivery system 100, sheath 180 is placed over shaft outer surface 172 such that folded portion 188 aligns with the matching groove 174 of shaft 150. Slit 190 may be cut into sheath 180 after or as it is placed into position on shaft 150. Slit 190 as disposed over shaft 150 such that the sheath folded portion 188 aligns with the folds of shaft groove 174 serves a strain relief function and eases assembly as the sheath 180 is disposed over shaft outer surface 172. Slit 190 also serves to help sheath 180, and, in particular, sheath distal portion 184, accommodate the larger shaft flared end 160 during delivery system 100 assembly. As assembled, delivery system 100 is configured in some embodiments such that sheath 180 covers both film 200 and the much of the length of shaft 150, including up to shaft flared end distal surface 160a on the distal portion 156 and at least up to and including a portion of shaft channel 166 near shaft proximal portion 152.

The properties of sheath 180, which may in various embodiments have a thickness of between about 0.50 mm and about 0.05 mm, between about 0.25 mm and about 0.10 mm, or about 0.15 mm, can be optimized to protect film 200 while affording the use of a small diameter shaft 150 (flaring to a larger diameter, as the compliant film may accommodate, at shaft distal portion 156). Having a small diameter shaft is useful in many procedures; including those in which access is limited in size and those in which an endoscope is used in the same lumen for visualization (such as is commonly done in sinus procedures).

Various aspects of shaft distal portion 156 (shown in, e.g., FIGS. 6A-6C) provide certain advantages in connection with the use of sheath 180. For instance, rails 162a and 162b on shaft distal tip 158 facilitate consistent and reliable axial retraction of the sheath 180 over film 200 and shaft outer surface 172 as the actuator 130 is moved by an operator in a proximal direction. The rails also help sheath 180 to keep fluid out of the vicinity of film 200 to avoid premature wetting thereof, and they can provide useful surfaces/extensions for moving and otherwise manipulating film 200 during and after its deployment to ensure its optimal adherence to the target tissue. As previously discussed, shaft flared end 160 also helps to shape sheath 180 as it is disposed over shaft 150 during assembly of the delivery system 100 to help ensure a uniform gap exists between sheath inner surface 192 and film 200.

Alternative sheath embodiments of the present technology are discussed below and include a sheath that is pinched at its distal end and otherwise sealed, completely or partially (e.g., perforated) via an adhesive, ultrasonic or thermal welding or the like, during delivery system assembly. During delivery system 100 use, as such a sheath is retracted axially in a proximal direction and shaft flared end distal surface 160a eventually meets the distal end of sheath, the shaft forces such a sheath to open at the seal/perforation, which may be designed to fail upon the application of such force. Such a design has the advantage of largely encapsulating the film 200 from the surrounding environment, providing additional protection against it coming into contact with fluids or humidity, thus improving film reliability during deployment and use. Another alternative embodiment for a sheath according to the present technology includes an encapsulated string that may be used to tear open a distal end of the sheath when it is proximally retracted. Yet another alternative embodiment for a sheath according to the present technology includes a biocompatible frangible wrapper design in which the sheath disintegrates or otherwise breaks apart at the intended temporal and physical location during the delivery of a film to the target tissue.

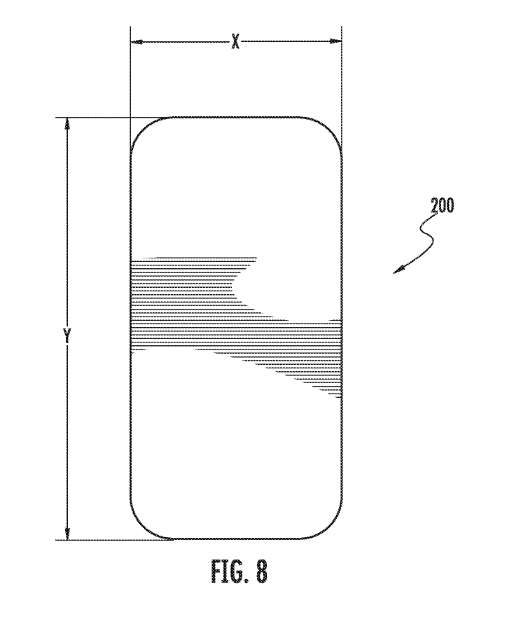

As assembled, delivery system 100 is configured to deliver one or more films 200 to a target location in a mammalian body for any number of therapeutic applications. In the sinus, such films 200 may be tailored to provide pharmaceutical treatment of iatrogenic wounds with therapeutic agents such as steroids. Other film configurations may be tailored to contain other compounds having specific formulae and dosage densities, as described in U.S. Pat. Nos. 8,529,941, 8,563,510, 9,072,681, 9,192,698, 9,271,925, and 9,446,103, to otherwise treat tissue in the sinuses and improve a patient's health and delivered via delivery system 100 in a safe and effective manner. For delivery system 100, film 200 and flared end 160 may generally be sized and shaped to match one another, as shown schematically in FIGS. 6C-6E to optimize performance of the film in vivo and its safe delivery to the intended target tissue location. For instance, for use in sinus applications and as seen in the example of FIG. 8, film 200 may be of a generally rectangular shape with rounded corners and have a surface area of between about 20 mm² and about 400 mm², between about 50 mm² and about 300 mm², between about 100 mm² and about 250 mm², between about 180 mm² and about 220 mm², or between about 190 mm² and about 205 mm². Accompanying width X and length Y values may match the ranges as described above for a given therapeutic agent density and configuration in the rectangular film configuration as shown in FIG. 8 and may respectively be, e.g., between about 2 mm and about 40 mm, between about 5 mm and about 30 mm, between about 8 mm and about 25 mm, or between about 10 mm and about 20 mm. With delivery system 100 for use in sinus applications, film 200 may have a thickness of between about 0.05 mm and 1.5 mm, between about 0.1 mm and about 1.0 mm, between about 0.3 mm and about 0.6 mm, or about 0.4 mm. Other thicknesses, single or multiple film layer configurations, surface areas, and widths/lengths may be used as necessary, depending on the indication for which the film is designed. The density, structure, and relative location to one another and within the film, be it of a single or multiple-layer construction, of the therapeutic agent(s) may be optimized to account for, e.g., any dimensional constraints or requirements imposed by delivery system 100 (including the desire to keep the diameter of shaft 150 as small as possible) and its intended use.

As previously described, FIGS. 1A-1B and 2 depict an embodiment of delivery system 100 in an assembled state and in exploded perspective, respectively, in accordance with the present technology. Handle 110 (including first half 116 and second half 118), shaft 150 and sheath 180, as well as actuator 130 are shown in general relation to one another in FIG. 2. Note in FIG. 2 the location of the distalmost portion 166a of shaft channel 166 relative to that of sheath post aperture 186. As assembled, shaft 150 fits within sheath lumen 194 such that sheath 180 is concentrically disposed over shaft 150 and sheath inner surface 192 is in direct contact with shaft outer surface 172 (except for the portion of shaft distal tip 158 where film 200 is disposed between the shaft outer surface 172 and sheath inner surface 192). Shaft 150 and sheath 180 are axially coextensive and rotationally aligned such that the sheath post aperture 186 is disposed directly over the distalmost portion 166a of channel 166. This allows actuator post or extension 140 to pass through aperture 186 into channel 166 during delivery system 100 assembly. This enables during use the axial movement of sheath 180 over the shaft outer surface 172 when an operator moves actuator 130 in a proximal direction so that film is exposed to the target body tissue and deployed thereon.

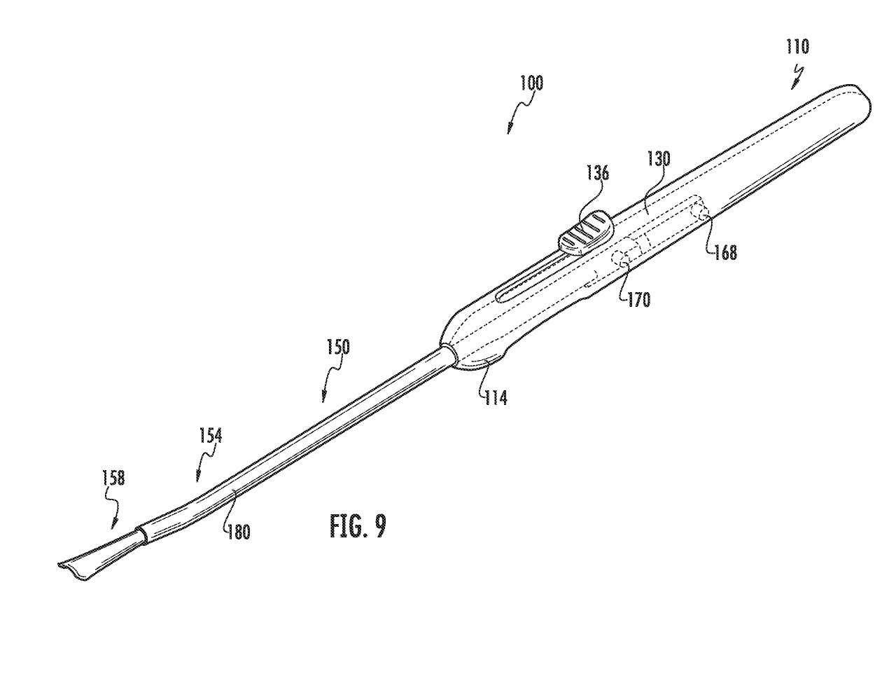

FIGS. 9 and 10 are partial cross section side and perspective views, respectively, of delivery system 100 as assembled. As shown in FIG. 1A, sheath 180 is disposed concentrically over shaft 150 and initially is coextensive on its distal end with the distal end of distal tip 158. This coextensive arrangement protects film 200, which is disposed between sheath 180 and shaft 150, from being prematurely exposed to bodily or other fluids and to prevent the premature deployment of film 200 until such time as desired. FIGS. 9 and 10 shows sheath 180 partially retracted to a greater extent than shown in FIG. 1B (by continued proximal motion of actuator 130), exposing shaft 150 distal portion 156 and in particular shaft distal tip/curved end effector 158 (film 200 is not shown).

Handle first half 116 and second half 118 enclose shaft proximal portion 152 and sheath proximal portion 182 that is disposed concentrically thereover. The handle halves are bonded and/or otherwise mechanically joined to one another by methods known by those in the art. Handle 110 is configured such that a handle central lumen 129 tapers from a first diameter in its proximal portion 112 to a second, smaller diameter in its distal portion 114 to enclose the shaft 150 and sheath 180. This serves to minimize lateral movement of shaft and sheath during storage. One or more optional seals (not shown) may be included in handle to facilitate a solid construction and to prevent fluid ingress into handle central lumen 129.

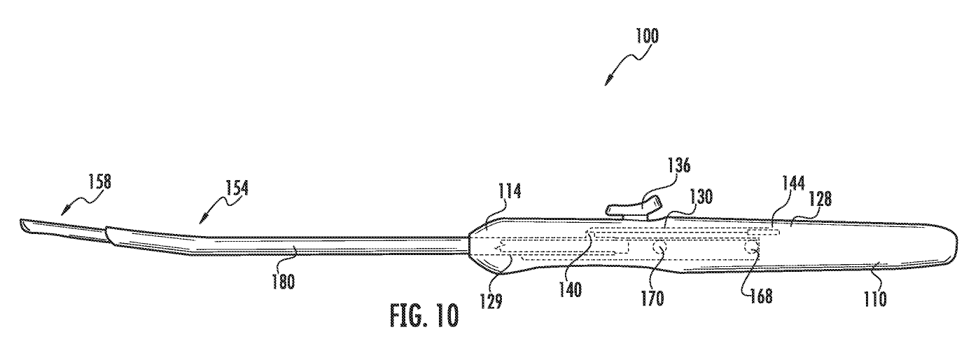

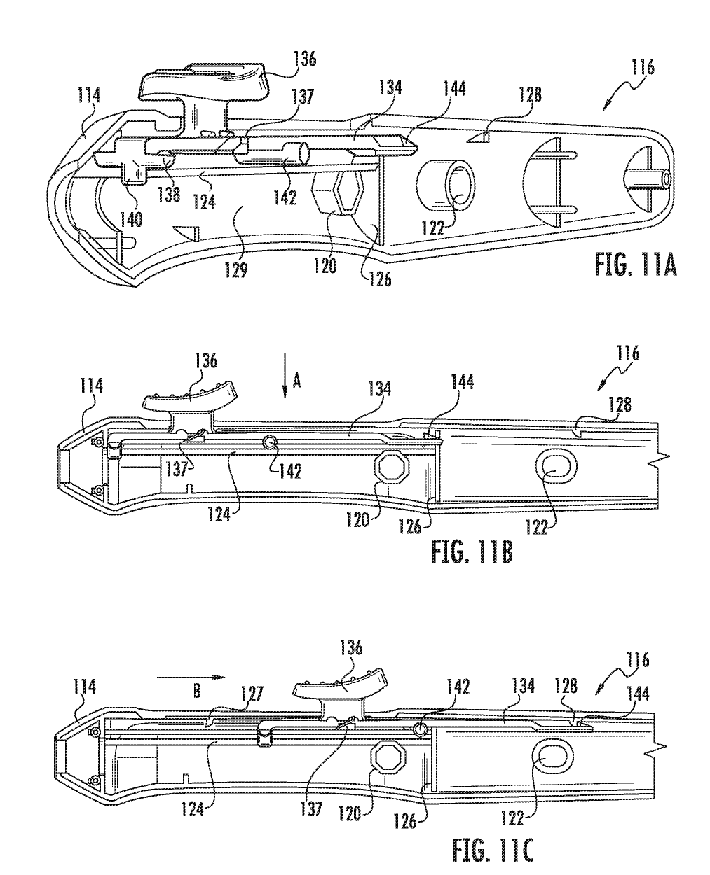

As shown in, e.g., FIGS. 12A-12B, shaft proximal peg 168 and distal peg 170 are received in proximal peg receiving shaft aperture 122 and distal peg receiving shaft aperture 120, respectively, in each of handle first half 116 and second half 118. As shown in FIGS. 11A-11B, handle distal peg receiving shaft aperture 120 has a generally hexagonal cross section while shaft distal peg 170 has a generally circular cross section. In addition, handle proximal peg receiving shaft aperture 122 has a generally elliptical or oval cross section while shaft proximal peg 168 has a generally circular cross section. The shaft distal and proximal pegs 170 and 168 are held in place within the distal peg and proximal peg receiving shaft apertures, 120 and 122, respectively, by an interference or friction “press” fit. Because of the dissimilar cross-sectional shapes between each shaft peg and its handle receiving shaft aperture, only a portion of a shaft peg's circumferential outer surface may make physical contact with the differently-shaped inner surface of the mating receiving shaft aperture, resulting in gaps therebetween. It may also result in a heterogeneous strain distribution around the circumference of a given shaft peg as it resides disposed in its respective shaft aperture. This design feature ensures simple yet secure fastening of the shaft 150 within handle 110 in a way that can provide both optimal mechanical compliance and stability. This in turn gives an operator excellent mechanical feedback during use. Other techniques for joining shaft 150 to handle 110 at these locations, such as adhesives, welding, etc. may be used alone or in combination with a press fit as described herein.

For sinus indications, the film deployment techniques using embodiments of delivery system 100 described herein typically are performed in conjunction with endoscopic visualization. Physicians skilled in endoscopic procedures expect a high degree of correlation between the operational performance of system 100 and what the operator sees via the endoscope. The construction of shaft 150 and handle 110 along with the other components of system 100 in embodiments of the present technology helps to ensure such correlation during use while at the same time providing enough overall system compliance such that delivery system 100 does not feel overly stiff to the operator or difficult to manage. The proper choice of materials for handle 110 and shaft 150 can also contribute to a delivery system 100 of the present technology that is optimized for its intended use.

Although shaft distal peg 170, handle distal peg receiving shaft aperture 120, shaft proximal peg 168 and handle proximal peg receiving shaft aperture 122 are described and shown as having particular cross-sectional shapes, other cross sectional shapes are within the scope of the present technology, including but not limited to various other shapes (e.g., pentagonal, octagonal, circular, elliptical, etc. as well as those shapes and dimensions varying throughout the width of the handle 110 halves and, accordingly, the length of the shaft 150 distal and proximal pegs.

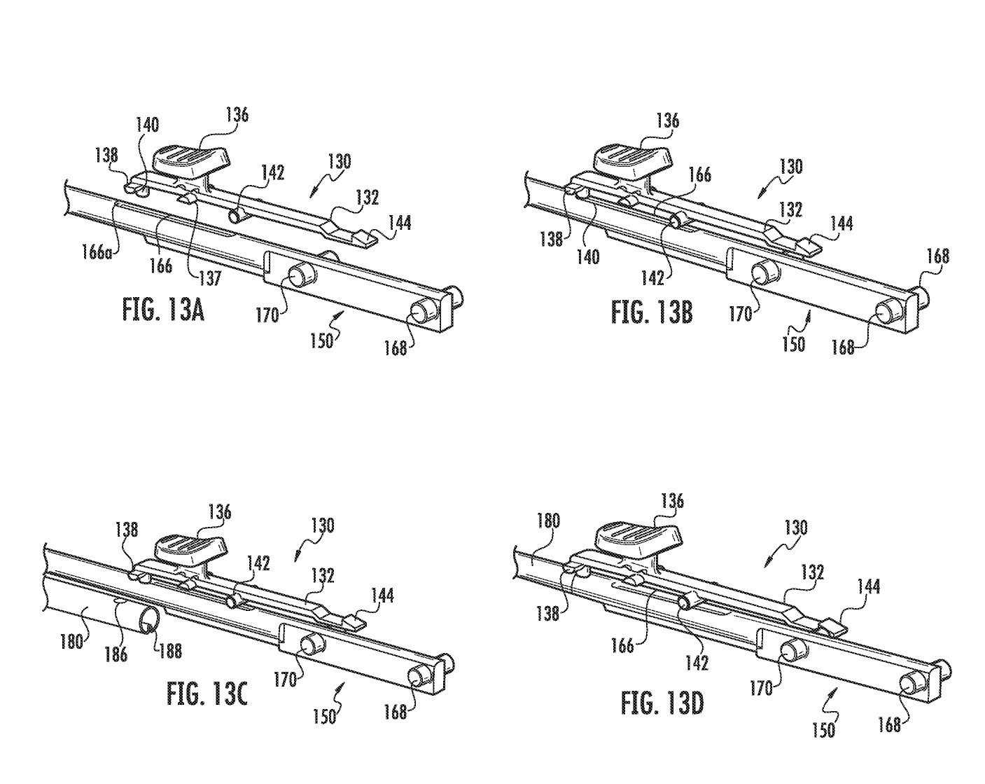

FIGS. 11B-11C depict actuator 130 of the present technology in partial cross section with handle first half 116. FIG. 11A provides a perspective view and FIGS. 11B-11C are elevational views in partial cross section showing two positions of actuator 130 relative to handle first half 116. Handle shelf 124 is configured to receive actuator distal boss 138 and proximal boss 142 so that upon axial motion of actuator 130 by an operator, actuator 130 slides along shelf 124 via contact with distal boss 138 and proximal boss 142. As previously described, and as shown in, e.g., FIGS. 10 and 13A-13D, sheath post aperture 186 and shaft channel 166 may together receive actuator post or extension 140 so that axial movement of actuator 130 in turn moves sheath 180 over shaft outer surface 172, eventually exposing film 200 for its deployment at a target body tissue site. Thumb pad 136 is disposed approximately midway between actuator distal boss 138 and proximal boss 142 (both supported on their underside by shelf 124) so to allow efficient flexure and downward movement (arrow A in FIG. 11B; actuator 130 is not shown being flexed in this direction in FIG. 11B) of actuator 130 upon the exertion of downward pressure on thumb pad 136 by an operator such as a physician. This downward movement A allows actuator tab 137 to move out of engagement with catch 127, thereby unlocking actuator 130 and allowing a user axially to move actuator 130 in a proximal direction. In this embodiment of delivery system 100, actuator tab 137 may be repositioned distal to catch 127, allowing it to be “relocked” into a position that prevents axial movement of actuator 130, giving the user operational flexibility during a film deployment procedure. Handle locking pawl or ramp 128 is configured to receive actuator locking tooth 144 disposed on the actuator proximal portion 132.

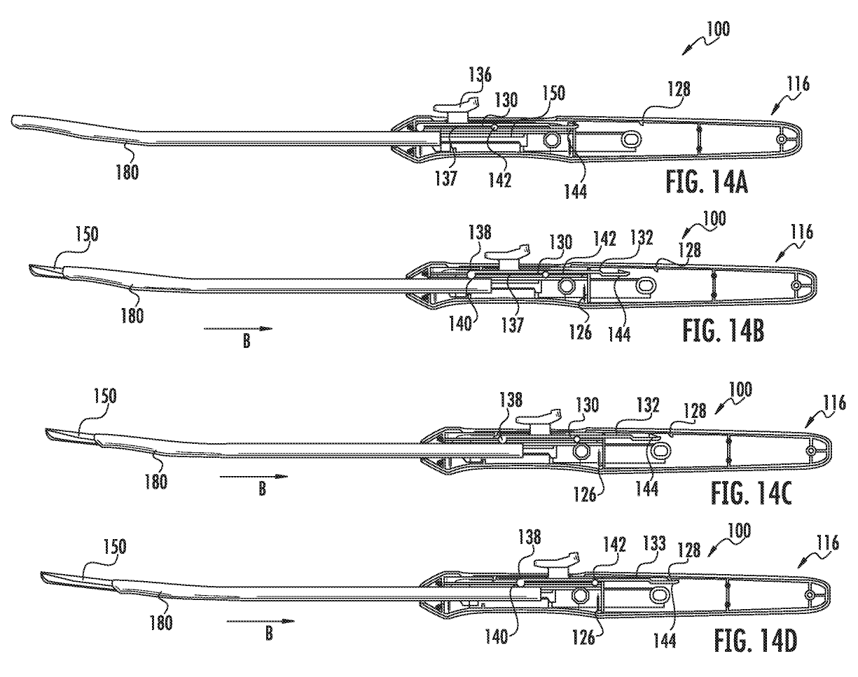

In operation, after a user has unlocked actuator tab 137 from handle catch 127 by exerting downward force on actuator 130 via thumb pad 136, the user will move actuator 130 axially in a proximal direction towards the proximal portion of handle 110, bringing sheath 180 with it along shaft outer surface 172. This axial movement is depicted in FIG. 11C and FIGS. 14B-14D by arrow B. As actuator continues to move proximally, actuator proximal boss 142 engages handle stop 126 to provide positive feedback to the operator that the actuator has exposed film 200 for deployment and to prevent further proximal motion of actuator 130 and sheath 180. In addition, as actuator locking tooth 144 engages handle locking pawl or ramp 128, actuator distal portion 134 is forced in a downward direction while the user continues to move actuator 130 in a proximal direction such that the angled surface of tooth 144 slides along ramp 128 until tooth 144 has moved to an axial location proximal to that of locking pawl 128 as shown in FIGS. 11C and 14D. At this point, actuator distal portion 134 has now flexed or moved in an upward direction, opposite of that shown in Arrow A of FIG. 11B, towards its natural unstressed state, such that a distal surface of tooth 144 engages a proximal surface of ramp 128 to prevent any distal axial movement of actuator 130. Any attempt by a physician user to unlock actuator 130 in a fashion similar to that to initially unlock it will not release ramp 128 from its engagement with tooth 144, thus rendering permanent the locking feature of actuator 130 within handle 110. These features are exemplary of any number of mechanisms that may be used in various configurations of the present technology to prevent distal axial movement of actuator 130 after film 200 has been exposed for deployment to the target tissue. This locking mechanism provides several advantages: it provides the physician operator tactile and perhaps audible feedback that the film 200 has been exposed for safe deployment to the target location, it prevents the physician-user from inadvertently moving the sheath in a distal direction and risking unsafe and/or undesirable interaction with the film 200 (that may have been partially deployed at this point), and provides for a single-use delivery system 100 with its attendant advantages from a product safety perspective. In addition, detents or other features (not shown) may be included in the actuator and/or handle to provide tactile and/or audible feedback to the user during proximal motion of actuator 130 indicating that a certain distance, e.g., 0.5, 1.0 or 2.0 cm, has been traversed, giving the user knowledge of how much further proximal axial travel is needed until the user is assured that film 200 is exposed for deployment.

Various materials known in the art may be used for the various components of system 100. Considerations such as biocompatibility, cost, amenability to sterilization, shipping, storage and performance may be taken into account when choosing a specific material. Such materials include chromium, cobalt, platinum and alloys thereof, stainless steel, with or without coatings such as PTFE, may be used. Plastics such as injection molded or extruded PET may be used, as may polycarbonates, PTFE, and combinations thereof. Gamma radiation-stable plastics include thermosets, polystyrene, LCP, polyurethanes, polyethylenes (PE, PET), polycarbonates, silicones, PVC, polyamides, ABS, acrylics such as PMMA, while plastics that may be sterilized via autoclave techniques include fluoropolymers such as PFTE, PFA, FEP, ePTFE, polypropylenes and their copolymers, polycarbonates, silicones, polyacetals, polymethylpentenes, polysulfones, synthetic elastomers and natural elastomers such as rubber.

Exemplary Method of Use.

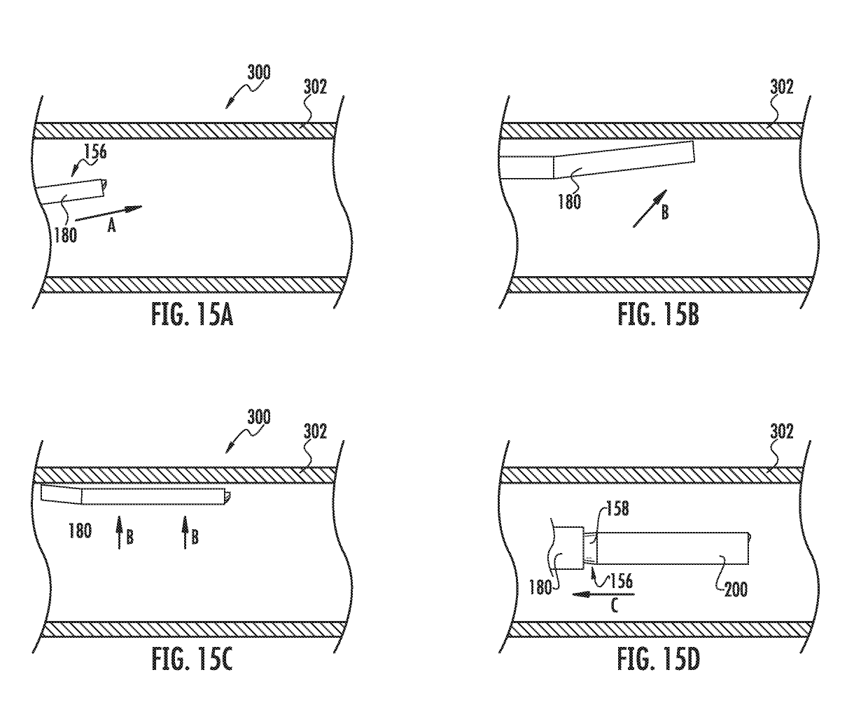

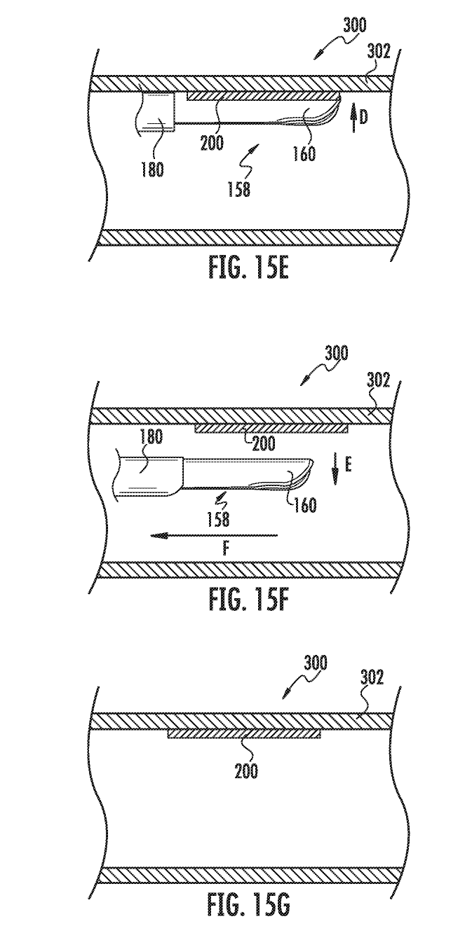

FIGS. 15A-15G illustrate a method of using film delivery system 100 configured in accordance with the present technology. Specifically, FIGS. 15A-15G illustrate a method of using film delivery system 100 to deploy or deliver one or more therapeutic films to the sinuses, such as, e.g., the ethmoid sinus. However, the illustrative method may be used to deliver one or more such films to other target tissue locations in the sinus as well as various other locations in a mammalian body as described herein. Modifications and additions to the delivery system 100 to tailor its use for a given targeted tissue region 300 are within the scope of the present disclosure, and include any number of shaft distal tip/end effector configurations as described below.

As previously described, FIGS. 1A-2 show delivery system 100 that includes handle 110, actuator 130, shaft 150 and sheath 180, and film 200 containing the desired therapeutic material having a density and dosage configuration specific to the indication for which the target tissue is to be treated. First, the physician-user will analyze the anatomical space of interest, e.g., the ethmoid sinus, and select a specific tissue region 300 target surface 302 for the placement of film 200.

After the patient is prepared using standard protocols for interventional sinus procedures, delivery system 100 is positioned via the handle such that the shaft 150 is inserted under endoscopic visualization or like means within and through a bodily access lumen, such as the nasal passages. Once the shaft distal portion 156 has been advanced to the target tissue region 300 as shown generally by arrow A in FIG. 15A, the physician can move shaft distal portion 156 to position it adjacent or against the target tissue surface 302 as illustrated generally by arrow B in FIGS. 15B-15C. Next, and after the physician is confident of the location of shaft distal portion 156 within the target tissue region 300 and in proximity of tissue target surface 302, the physician unlocks actuator or slider 130. In the embodiments of the present technology, this is accomplished by the physician applying a downward force on the actuator thumb pad 138 as previously described. Once the actuator or slider 130 is unlocked, and as guided if desired under endoscopic visualization or like means, the physician may axially move the actuator or slider 130 axially towards proximal portion 112 of handle 110. As the actuator or slider 130 is so retracted within handle 110, actuator post or extension 140 slides axially in a proximal direction within shaft channel 166, engaging surface 186a defining sheath post aperture 186 and moving sheath 186 in a proximal direction along with actuator 130. As the actuator or slider 130 continues to be retracted axially, optional detent structures or similar features (not shown) in the handle 100, shaft 150 and/or actuator may provide tactile and/or audible feedback or signals to the physician user that a specific distance has been traversed as previously described. During sheath retraction as illustrated generally by arrow C in FIG. 15D, film 200 is gradually exposed to the environment of the body tissue target region 300 and tissue surface 302, including exposure to any bodily fluid such as blood that may be in the field at the target tissue location region 300. Such exposure may activate certain therapeutic compounds within the film and/or change the physical characteristics of the film. In addition, such exposure may “wet” film 200 such that it may be readily deployed from the shaft outer surface 172 at shaft distal end 156 and adhered to the target tissue.

As the actuator or slider 130 is axially retracted in a proximal direction to its final position, the locking mechanism of the handle locking pawl or ramp 128 and actuator locking tooth 144 engages actuator so that actuator 130 may not be moved in a distal direction. Actuator proximal boss 142 engages handle stop 126 to prevent further axial motion of actuator 130 and sheath 180 in a proximal direction as previously described. The physician is then free to manipulate delivery system 100 under visualization to release film 200 to the target tissue. This typically may be accomplished as the physician manipulates the shaft distal portion 156 physically to place film 200 into direct contact with target tissue surface 302 as illustrated generally by arrow D in FIG. 15E. When this is accomplished, the wetting effect of the film having been exposed to fluids and resultant adhesion forces between the film 200 and target tissue surface 302 are sufficient to overcome any forces retaining film 200 on shaft outer surface 172. This results in the gentle release, delivery or deployment of film 200 from the shaft outer surface 172 onto the target tissue surface 302 as shown in, e.g., FIG. 15F. Although it is not shown in the figures, if desirable and/or necessary, the physician may use the shaft distal tip 158, including the flared end 160 and optional rails 162a, 162b, to manipulate and/or move film 200 into an optimal position on the target tissue surface and to ensure optimal contact therewith. Delivery system 100 may then be removed from the target tissue region 300 under visualization via the same bodily lumen through which access was gained. This is generally illustrated by arrows E and F in FIG. 15F, with FIG. 15G illustrating film 200 remaining adhered to target tissue surface 302 in region 300 with system 100 withdrawn.

Additional films 200 may be delivered by additional delivery systems 100. For procedures in the ethmoid sinus, for example, between one and two or more additional films optionally may be deployed at a single target tissue location or at other target tissue locations in the ethmoid or other sinuses.

Although described as configured for the delivery of a single film, delivery systems of the present disclosure may be configured with multiple films 200 such that a single delivery system 100 may deliver two, three, four or more films in a single procedure.

II. Alternative Film Delivery Systems and Associated Methods

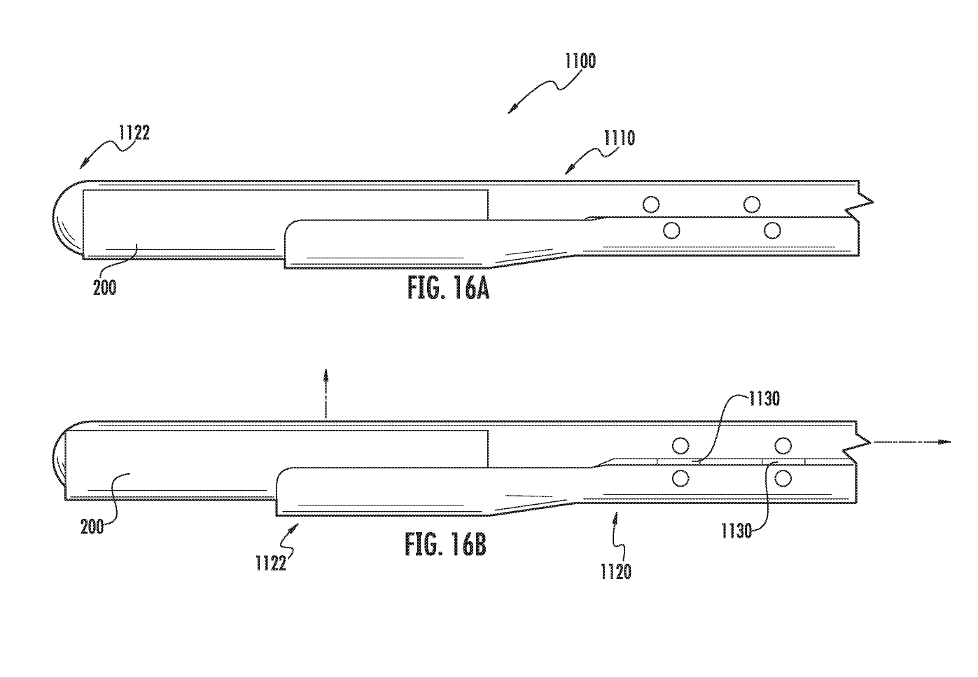

FIGS. 16A-30B depict various alternative film delivery systems and associated methods, including a number of alternative shaft distal tip/end effector designs for releasing one or more films 200 according to the present technology. These alternative system are described below in more detail. As will be appreciated by those of skill in the art, each of these alternative delivery system shaft distal tip/end effector configurations may be used with the components as described herein, perhaps with modifications, to accommodate the end effector design of interest and the target tissue to which the film is to be delivered. Each of the various embodiments described herein with respect to Figures 1A-30B may be more suitable for use in a particular part of the body, and/or for different indications, in comparison to other embodiments. For instance, the embodiments of FIGS. 1A-30B are particularly suitable for use in treating sinus tissue as discussed above, although the embodiments of FIGS. 1A-30B may be used in other parts of the body to treat different indications.

Modified Forceps Film Delivery System

FIGS. 16A-16B depicts a distal portion of an alternative, modified forceps film applicator 1100 in which deployment of film 200 is accomplished by the motion of first upper arm 1110 relative to second lower arm 1120 between which film 200 is disposed. In the embodiment shown, arms 1110 and 1120 have atraumatic curved first and second element distal portions 1112 and 1122, respectively. Arms 1110 and 1120 are connected by hinges 1130. Prior to deployment of film 200, arms 110 and 1120 are biased in a closed position to clamp or hold film 200 therebetween for delivery to the target tissue region 300 of interest.

As shown in FIG. 16B, the design of the modified forceps delivery system 1100 allows for the release of film 200 when a physician or other operator effects relative motion between arms 1110 and 1120. This is depicted here by the exemplary movement of upper arm 1110 in the proximal direction indicated by arrow A and the upward direction by arrow B, with lower arm 1120 remaining stationary). The upward movement B is made possible by the offset X present between elements of hinges 1130 in upper arm 1110 and lower arm 1120 (shown here as pins). In particular, when a physician moves upper arm 1110 proximally, the proximal rotation of hinges 1130 forces upper arm 1110 upward by a distance Y as the offset X is reduced or eliminated. This pivot of upper arm 1110 in turn frees film 200 to slide out to be deployed by the physician onto the target tissue surface 302. Film 200 distal portion 202 is only partially covered by lower arm distal portion 1122 in the embodiment of FIG. 16 so to allow pre-placement and/or adherent contact of film 200 onto the target tissue surface 302, facilitating a more reliable and precise deployment of film at the intended location at the desired time. Upper arm 1110 (and/or, e.g., lower arm 1120) may also act as a secondary repositioning and/or release feature as an operator may move upper arm 1110 in a distal direction to re-clamp film 200 if desired to reposition film 200. The embodiment of FIG. 16 is amenable to endoscopic visualization and facilitates a reliable film release for indications in which a low profile is important or desirable. Further, the embodiment of FIG. 16 is well-suited as a reusable device; alternatively, it may be employed in single use applications. Other elements of system 1100 (e.g., a shaft, proximal handle, grip elements) may be incorporated as known by those of skill in the art. And while the embodiment of FIGS. 16A-B shows a hinged design incorporating pins, other designs that allow the release of a film by the motion described herein, both locking and non-locking, may be used.

In a method of use of the embodiment of applicator 1100 shown, once the physician or other operator accesses the target tissue region 300 of interest, he or she will effect proximal motion of upper arm 1110 as described above, pivoting it so to free film 200 such that is deployed onto the target tissue surface 302. The physician or other operator may use upper arm 1110 as a secondary repositioning and/or release feature, moving upper arm 1110 in a distal direction to re-clamp film 200 if desired to reposition film 200. After film deployment, the physician or other operator may withdraw applicator 1100 and optionally deploy additional films with additional applicators as desired.

Sponge Tip Applicator Film Delivery System

A perspective view of a distal portion of another alternative delivery system embodiment of the present technology is shown in FIG. 17 in the form of a sponge tip applicator 1200. Applicator 1200 shown in FIG. 17 includes shaft 1210 having a central lumen or passageway 1220 configured to be in fluid connection with a source of fluid, such as saline or water. One or more apertures 1230 in fluid connection with lumen 1220 are disposed at distal end 1212 of the shaft. A sponge or foam element 1240, which may be medical grade and of open-cell construction, is disposed to surround shaft distal end 1212 in the vicinity of apertures 1230. In this embodiment, film 200 is disposed on the bottom of sponge 1240, matches the curvature of the bottom surface of sponge 1240 and is sized to match the length of sponge. Sponge 1240 may be pre-wet in order to adhere better to film 200 and may take on any number of lengths, cross-sectional shapes, dimensions, material configurations (e.g., composite construction), etc. to optimally suit the intended clinical use. In the embodiment of FIG. 17, apertures 1230 are aligned with the bottom (film side) of shaft 1210 to effect an efficient film release, but other configurations in which the apertures 1230 are located in different positions on shaft 1210, have different sizes, differ in number and orientation relative to one another, have different and/or varying opening profiles (e.g., elliptical, slit, etc.) are contemplated for use in the sponge tip applicator embodiment 1200. For instance, apertures of increasing diameter as they are disposed distally on shaft 1210 may be useful to ensure uniform release of fluid due to pressure gradients that naturally occur as one moves towards shaft distal end 1212.

In a method of use of applicator 1200, once the physician or other operator accesses the target tissue region 300 of interest, a biocompatible fluid (e.g., water, saline, solutions containing one or more therapeutic substances, additives to control the film-sponge adhesion and release properties and timing, etc.) is introduced via optional pressure thorough passageway 1220. This may be accomplished any number of ways, including via a syringe connected by a luer or other fitting to a catheter that is connected to or part of shaft 1210, or via an Endoflator® or other device to control to pressure of fluid being delivered, etc. As the fluid enters the vicinity of the shaft distal end 1212, it flows through apertures 1230 and/or an optional open end of shaft 1210 to wet sponge 1240. As sponge 1240 becomes saturated with the fluid, particularly in the vicinity of film 200, the adherence between sponge 1240 and film 200 decreases to the point that when desired, the physician or other operator may deploy film 200 at the tissue target surface 302 via application of pressure and movement of shaft 1210 in the manners as described herein for other embodiments of the disclosure. After film deployment, the physician or other operator may withdraw applicator 1200 and optionally deploy additional films with additional applicators as desired.

As may also be accomplished with respect to the modified forceps delivery system 1100 and all the delivery system embodiments depicted herein, the sponge tip applicator system 1200 may be used for any number of film configurations, including those in which an therapeutic substance is largely on the side of film 200 adhering to sponge (given that once deployed, this side of film 200 will be exposed to, e.g., blood flow or the lumen of the passageway or bodily cavity in which it is deployed) and a different substance such as fibrinogen or combination of substances is largely on the opposite side of film 200 (given that once deployed, this opposite side of film 200 will be in direct contact with and intended to adhere to/incorporate with tissue in the region of target tissue surface 302.

Wireform Release Applicator Film Delivery System

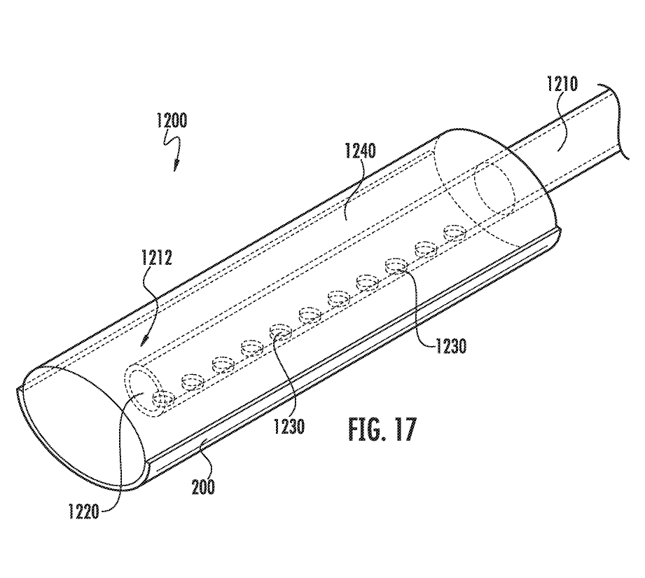

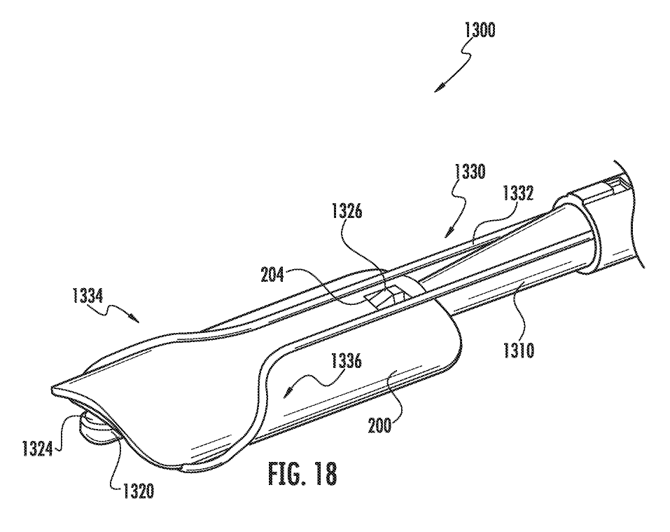

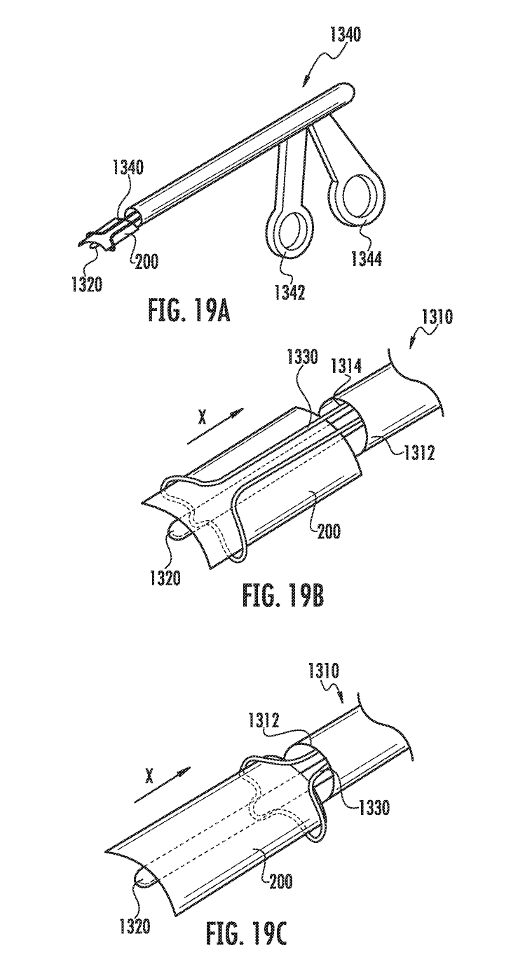

FIGS. 18-19C depict a further embodiment of the present disclosure in the form of a wireform release applicator 1300. As seen in FIG. 18, applicator 1300 can include shaft 1310 and wireform 1320 that may be involved with holding film 200 in place and releasing it into the body at the target tissue region 300 as described herein.

Shaft 1310, which may be made from any biocompatible material such as molded plastic or, e.g., a metallic material such as stainless steel, may be a part of a tool 1340 shown in FIG. 19A for use by an operator to release film 200 as desired. Shaft distal tip 1312 may be configured to accommodate probe 1320 connected thereto.

Probe 1320, which is shown in FIGS. 18-19C as having a tapered configuration, may have film surface 1324 designed to accommodate and hold in place film 200, with or without optional retaining tab. In use, probe may be used by a physician or other operator to position or reposition film 200 at the target tissue surface 302.

Wireform 1330 is a retractable guide that serves several functions, including holding the film 200 in place against probe film surface 1324, retracting to reveal film 200 at the desired target tissue region 300 for release and placement on tissue surface 302, and can be used to separate film 200 from probe 1320 if necessary (when, e.g., film 200 will not freely release from probe film surface 1324). Wireform 1330 may be made of any suitable medical grade material, such as stainless steel, plastic, or a shape memory material such as NiTi. Wireform 1330 may take on any number of shapes configured for optimal retention of and deployment of film.

In one configuration as shown in FIGS. 18-19C, wireform 1330 includes extruded wire 1332 having loop 1334 on its distal end and flare feature 1336 that accommodates partial passage therethrough of a portion of film 200 (slightly different configurations for wireform 1330 are shown in the embodiments of FIGS. 18 and 19A-C). As shown in FIG. 18, optional retaining tab 1326 may be passed through an optional aperture 204 on film to aid in retaining film on probe film surface 1324 until such time as deployment of film 200 is desired. At the juncture of probe 1320 and shaft 1310, wireform 1330 is disposed within shaft lumen 1314 and may be connected in any number of ways to a mechanism to allow an operator to retract wireform 1330 to release film 200. In the embodiment of FIG. 19A, one such retraction mechanism is depicted. Here, tool 1340 includes first and second handles 1342, 1344, as known by those of skill in the art, which may be moved together or apart relative to one another by a physician or other operator to retract wireform 1330 and release film 200.

In a method of use of this embodiment shown in FIGS. 18-19C, handle 1342 may be connected to a proximal portion or end (not shown) of wireform 1330 and squeezed by a physician or other operator in triggerlike fashion towards handle 1344 so to retract wireform in a proximal direction X towards handles 1342, 1344 and reveal film 200 as further depicted in FIGS. 19B and 19C. If retaining tab 1326 is present, as film 200 is freed to be deployed at the target tissue site 302. Optionally, further retraction of wireform 1330 (which may be effected by a detent or other two-step mechanism to indicate to the physician or other operator of the location of the wireform 1330 relative to film 200 and shaft 1310) will cause wireform loop 1334 and flare end 1336 to deform and allow the partial or full retraction of wireform 1330 into shaft lumen 1314 at shaft distal tip 1312. This feature is particularly suited to shape memory materials, such as shape memory polymers or NiTi or other metallic alloys known to those of skill in the art, to facilitate deformation into shaft lumen 1314 and, if desired, redeployment in the opposite direction outside of shaft lumen 1314, in which case wireform loop 1334 and flare end 1336 may take on its original shape or configuration. This is advantageous if wireform release applicator 1300 is desired to deploy multiple films 200 in a single procedure, as the physician or other operator may load any number of additional films onto probe film surface 1324 and deploy them to target tissue surface 304 as desired. Wireform 1330 may be designed to have optimal stiffness and other properties such that a highly controllable and reliable film retention and release mechanism, with a large degree of precision feedback and “feel” for the physician or other operator, is possible while minimizing the volume of hardware necessary to deploy film 200 to optimize visualization.

For the wireform release applicator embodiment 1300 described herein, as with the other applicator embodiments disclosed, film 200 may be configured such that a therapeutic substance is disposed on the side of the film facing probe film surface 1324, while one or more other substances (such as fibrinogen) may be disposed on the opposite side of the film.

Rotating Tine Film Delivery System

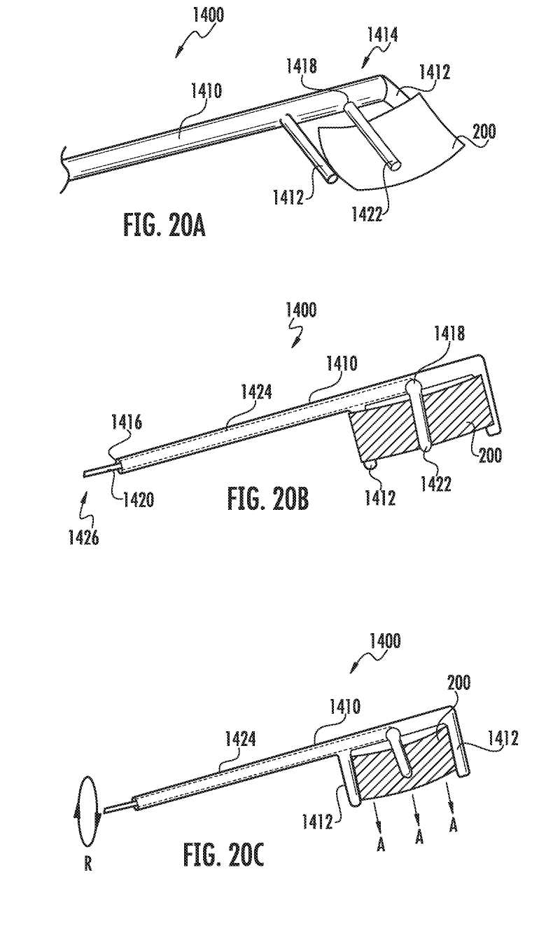

FIGS. 20A-C illustrates a film delivery system that utilizes rotating tine applicator 1400. Applicator 1400 may include a hollow outer shaft 1410 to which two or more stationary tines 1412 may be fixed at shaft distal end 1414. A rigid inner shaft 1420 is disposed within lumen 1416 of outer shaft 1410 to which at least one rotating tine 1422 is attached.

Outer shaft 1410 may be of a plastic or metallic (e.g., stainless steel) construction. In the embodiment of FIG. 20, two stationary tines 1412 are shown integrated with shaft 1410 at outer shaft distal end 1414. Stationary tines 1412 may be attached to shaft 1410 by known means, such as welding, brazing, and the like, or they may be an integral feature of shaft 1410 by being molded or formed as a single unit with shaft 1410. At least one aperture or slot 1418 is disposed on outer shaft distal end 1414 for the passage therethrough of a rotating tine 1422. Slot 1418 may be sized so to allow rotating tine 1422 to rotate, upon rotation of inner shaft 1420 by a physician or other operator as shown in FIG. 20C by arrow R, therewithin in a stable manner through a degree of rotation as may be desired, such as between about 5 degrees to about 180 degrees.

Tines 1412 generally are of uniform length and diameter and orientation relative to shaft 1410. Stationary tines 1412 are spaced from one another and have lengths that optimally support a film 200, as shown in FIG. 20A-C in which their spacing is generally similar or the same as of the length Y of film 200 (see, e.g., FIG. 8) and their length is on the dimension of the width X of film 200. One or more additional stationary tines 1412 may be included to provide further support for film 200 as necessary for the film's safe deployment at the body target tissue site or region 300. Stationary tines 1412 are shown in FIGS. 20A-C as having a circular cross-sectional shape, but they may have any cross-sectional shape as optimized for film 200 deployment. For example, one or more of stationary tines 1412 may have a generally flat surface (not shown) on their side intended for receiving film 200 to provide a larger surface area to support film 200, such as by use of a partially circular cross-sectional shape or a square, rectangular, etc. cross-sectional shape.

Disposed within lumen 1416 of outer shaft 1410 is a rigid inner shaft 1420, shown as extending through outer shaft 1410 in FIGS. 20B-C by dotted lines. As described above with respect to outer shaft 1410, inner shaft 1420 may be made of plastic or be metallic (e.g., stainless steel) and at least one rotating tine 1422 may be attached to or integrally formed as a part of inner shaft 1420 at inner shaft distal end 1424. In some embodiments, inner shaft 1420 is made from a high-stiffness steel, nitinol, or other suitable material to provide optimal response and torqueabililty for the physician or other operator when using applicator 1400. As assembled, the at least one rotating tine 1422 is disposed through an aperture or slot 1418 in outer shaft 1410 to allow tine 1422 to move and release film 200 as inner shaft 1420 is rotated within the outer shaft lumen 1416. Outer shaft 1410 may be of two-part construction so to facilitate creation of aperture 1418 or aperture 1418 may be created by, e.g., laser drilling, machining, chemical etching, etc. on a single extrusion of hollow outer shaft 1410.

A method of using applicator 1400 includes a physician or other operator deploying applicator 1400 to a target body tissue site 300 of interest, under optional visualization. Film 200 is loaded onto applicator 1400 as shown in FIG. 20A such that it is deployed on and in contact with one side of stationary tines 1412, such their upper surface, and under and in contact with one side of rotating tine 1422, such as its lower surface. In this manner, film 200 is secured for delivery to the tissue region 300 until such time as it is to be deployed on tissue surface 302. When deployment of film 200 is desired, the physician or other operator holds outer shaft 1410 stationary while rotating inner shaft 1420, at a proximal region 1426, in a direction R to effect rotation of rotating tine 1422 within slot or aperture 1418. This rotation is illustrated in FIG. 20B. Inner shaft 1420, and with it rotating tine 1422, may be rotated by any degree necessary to effect film deployment as provided by applicator 1400, such as, e.g., through an angle of rotation ranging from about 5 degrees to about 180 degrees.

In one embodiment as shown in FIG. 20B, rotating tine 1422 may be rotated as described above in a clockwise manner towards a target tissue surface 302 (not shown), pushing film 200 past stationary tines 142 in a direction A such that a bottom surface of film 200 is free to be placed against tissue surface 302 by the action of shaft/rotating tine rotation. Once deployed, rotating tine 1422 (as well as other elements of applicator 1400 at outer shaft distal end 1414) may be used to secure film 200 or to reposition film 200 as desired. To facilitate film 200 deployment, a surface of either or both film 200 and rotating tine 1422 may be pretreated so to allow a degree of adherence between rotating tine 1422 and film 200 to improve the deployment sequence reliability and effectiveness. In addition, the spacing of stationary tines 1412 relative to film 200 may be optimized to provide secure disposition of film 200 thereon but a reliable deployment action such that edges of film 200 may move past tines 1422 as rotating tine 1422 is rotated.

Applicator 1400 is amenable to deployment of multiple films 200 in a single procedure due to the relative ease with which film 200 may be loaded thereon.

For the rotating tine applicator embodiment 1400 described herein, as with the other applicator embodiments disclosed, film 200 may be configured such that a therapeutic substance is disposed on the side of the film facing rotating tine 1422, while one or more other substances (such as fibrinogen) may be disposed on the opposite side of the film.

Suction Tip Applicator Film Delivery System

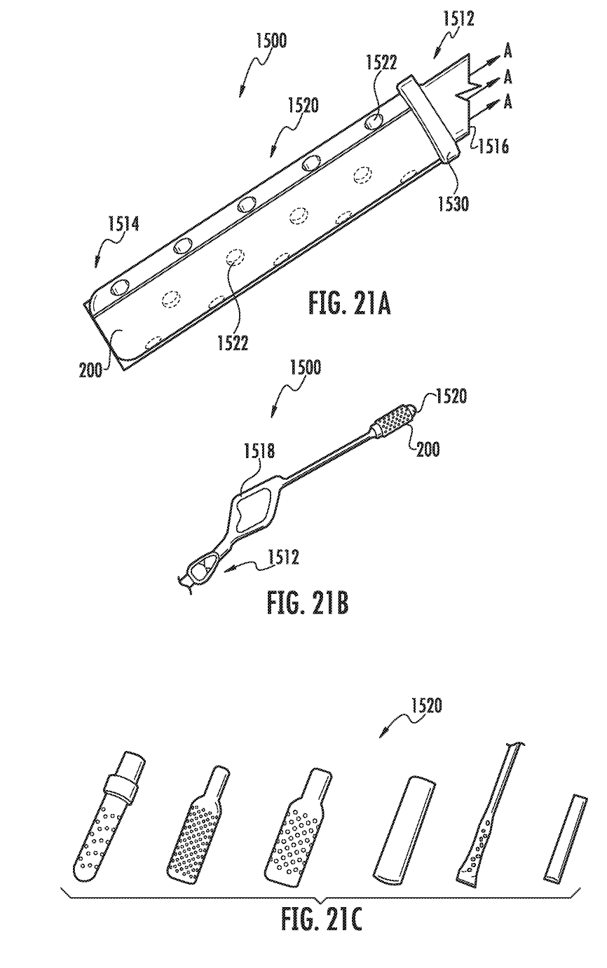

FIGS. 21A-21C depict another film delivery system in the form of a suction tip applicator 1500. Applicator 1500 may include hollow shaft 1510 that at proximal end 1512 may be connected to or a part of a surgical suite's suction system or to a dedicated source of suction (such as, e.g., would be a part of applicator 1500). Applicator 1500 may also include tip 1520 at shaft distal end 1514 onto which a film 200 is deployed for release onto the target tissue surface 302.

Hollow shaft 1510 may be a shaft of rigid construction, made, e.g., of plastic, metallic or other material. Shaft 1510 proximal end 1512 can have a fitting or adapter that allows for its connection to a surgical suction system, such as via a clamping mechanism. Shaft proximal end 1512 may in other embodiments be connected to a different, dedicated source of suction designed for use with applicator 1500, such as, e.g., a self-contained portable or semi-portable vacuum system that may be handheld or even battery operated, that is part of applicator 1500 such as in a kit. In any configuration contemplated for the embodiment of application 1500, an optional handle 1518 (FIG. 21B) with optional suction controls (such as, e.g., a suction on-off switch, suction power dial or buttons, indicator lights or other signals, user interface, etc.) may be incorporated into applicator 1500, to facilitate manipulation and use during a film deployment method or procedure as discussed below. Shaft inner lumen 1516 is in fluid communication with whatever source of suction to which it is connected as well as to a tip inner lumen (not shown), as described below.

Tip 1520 may be of a flat, circular or other shape amenable to the indication for which it would be used in the body region of interest 300. It may take on the shape of, e.g., a canoe paddle end or it may be a more streamlined design with an outer diameter similar or identical to that of shaft 1510 to which it is attached or is a part. Various examples of tip 1520 are shown in FIG. 21C. Tip 1520 may be made from metallic, plastic, or composite material as desired. Disposed on tip 1520 is at least one suction aperture 1522 in fluid connection with an inner lumen 1524 of tip 1520. In the examples of FIGS. 21A-21C, multiple suction apertures 1522 of various configurations and sizes are shown and are contemplated by the present technology. Inner lumen 1524 is in turn in fluid communication with hollow shaft inner lumen 1516. If tip 1520 is designed as an integral part of shaft 1510, tip inner lumen 1524 can be thought of as a distal end section of shaft inner lumen 1516; alternatively, tip 1520 may be configured to be attached to hollow shaft distal end 1514 either during manufacturing or, as may be desired, during or just before a film deployment procedure. In any configuration, inner lumen 1524 will be in fluid communication with a suction source (not shown) via its connection to shaft inner lumen 1516. An advantageous feature of applicator 1500 is that it may be made available as a kit such that any number of tips 1520 of various sizes, shapes, and configurations suitable to the indication of interest and the film to be deployed may be used in an interchangeable fashion by the physician or other operator.

An optional shoulder 1530 may be incorporated into applicator 1500 for providing an additional mechanism by which the physician or other operator can deploy film 200. For example, shoulder 1530 may be a stationary feature that serves mainly to protect the back edge of film 200, as shown in FIG. 21A, from damage during the deployment method or procedure. In other embodiments, shoulder 1530 may be an active component of applicator 1500 to help force, or push, applicator onto the target tissue surface 302 after suction is stopped or reduced during the film deployment procedure. This may be accomplished by, e.g., shoulder 1530 being connected to or being an integral part of shaft distal end 1514, wherein shaft 1510 is concentrically disposed on, and slidable over, a separate hollow shaft that is connected to tip 1520. This separate hollow shaft (not shown) may have an inner lumen for fluid connection to the tip inner lumen 1524 and a source of suction so that film may be adhered during operation by suction force to tip 1520.

In use, a physician or other operator configures applicator 1500 with the desired type of tip 1520 amenable to the procedure for which it is to be used. Alternatively, the physician or other operator may select a system 1500 that includes a pre-attached or even integral tip 1520, complete with an optional dedicated source of suction as described above. The physician or other operator connects shaft 1510 to a source of suction, if so configured, that may be present in the surgical suite, or activates a dedicated source of suction in the alternative configuration described above. The activation of suction through the components of applicator 1500 provides a vacuum force (indicated in FIG. 21A by arrows A) that causes film 200 to adhere to tip 1520 near tip apertures 1522. Under optional visualization, the physician or other operator then accesses the tissue site of interest 300. When such site is reached, the physician may manipulate the applicator 1500 such that film 200 is on or near the tissue surface 302, and the suction source may be reduced (via a variable control) or turned off so to release film 200 to tissue surface 302 in a controllable manner. If shoulder 1530 is present on shaft 1510, the physician may slide or otherwise manipulate the shaft in a distal direction to assist the controlled release of film 200 from tip 1520. In circumstances in which the suction is intentionally reduced but not eliminated, use of shoulder 1530 serves to provide additional precise control during film deployment. In circumstances in which the film may still adhere to tip 1520 after, e.g., suction is largely or completely eliminated, use of shoulder 1530 provides a back-up mechanism to force film 200 in a controlled manner that does not damage film or its therapeutic contents off of tip 1520 and onto the target tissue 320.

If movement or repositioning of the film is desired, suction may be reintroduced to pull film 200 back on to tip 1520, giving the physician or other operator a convenient means by which the film may be optimally placed.

For the suction tip applicator embodiment 1500 described herein, as with the other applicator embodiments disclosed, film 200 may be configured such that a therapeutic substance is disposed on the side of the film facing tip 1520, while one or more other substances (such as fibrinogen) may be disposed on the opposite side of the film.

Passive Applicator Film Delivery System

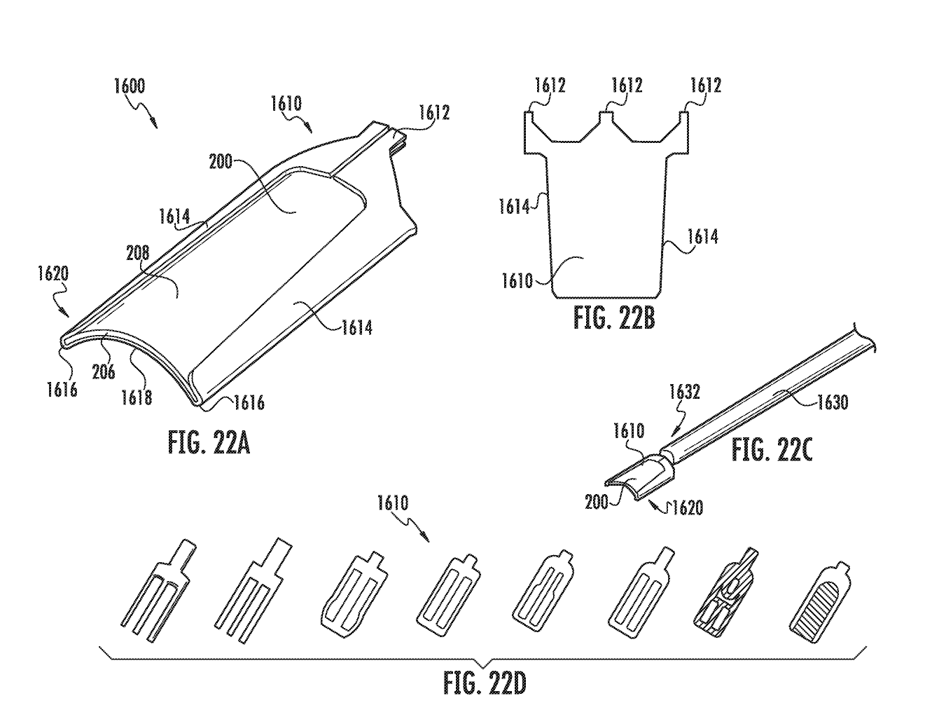

FIGS. 22A-22D depict another film delivery system in the form of a passive applicator 1600. Applicator 1600 may include an applicator tip 1610, typically in the form of a disposable molded plastic or metallic component that intended to be shipped from the manufacturer as pre-assembled with film 200 incorporated therein. An optional handle 1630 may be included as described below. Applicator tip 1610 may come in a variety of shapes, sizes, and materials optimized for the indication for which it is designed as shown in the examples of FIG. 22D. For instance, the tip embodiment shown in FIGS. 22A-22C and some of the embodiments of FIG. 22D, may be well-suited for sinus indications, given that tip 1610 may be formed as a clear or opaque member that is curved to match the curvature of the sinus cavity tissue 300 into which film 200 is to be deployed. In particular, and as shown in a flat pattern view in FIG. 22B, tip 1610 includes a proximal grab point 1612, a feature optimized for gripping by a surgical instrument such as pair of forceps, as illustrated in FIG. 22A, for delivery to target tissue region of interest 300. As shown in the flat pattern view of FIG. 22B, this embodiment of tip 1610 may be die-cut from a sheet of suitable biocompatible material, such as a low-friction thermoplastic material suitable to heat forming. Tip 1610, after being cut, may be folded into the configuration shown in FIG. 22A and heat-welded or compacted at proximal grab point 1612 and optionally along one or both edges 1614, and formed into the desired shape, such as a curved shape optimized for the tissue and cavity into which it will be deployed.

Film 200 may be shaped and sized to fit the dimensions of tip 1610 and may be assembled and integrated into tip 1610 at the manufacturing site or alternatively by the customer or end user, including the physician or other operator. Edges 1614 of tip 1610 are folded over so to form a compartment or channel 1616 into and out of which film 200 slides during assembly and deployment, respectively. Film 200 is supported on its underside 206 by tip main portion 1618. It is useful to size and assemble the folded-over portions of tip edges 1614 in a way to securely hold allow secure holding of film 200 in place until it is to be deployed, but to allow for a smooth and reliable film deployment procedure at the desired time, as film 200 slides out of tip 1610 in the manner described in the method example below. In addition, choosing a low-friction material to serve as tip 1610 will aid in a smooth film deployment method.

An alternative configuration includes a tip 1610 having a shaft or handle 1630, as shown in FIG. 22C. Handle 1630 may be a separate component or integrally formed as part of tip 1610. In this configuration, a distal end 1632 of handle may be connected to another handle or component or may be held directly by the physician or other operator, depending on the indication that is being treated.

A method of use for applicator 1600 may be accomplished as follows: tip 1610, having film 200 preassembled therewithin, is placed at proximal grab point 1612 by a physician or other operator by forceps or other suitable surgical instrument, with film 200 optionally being wetted prior to placement in the target tissue region 300. Under optional visualization, the physician or other operator places the tip 1610 with included film 200 near the target tissue surface 302 such that film top surface 208 faces tissue surface 302. The physician or other operator, once satisfied with the position of the tip 1610 and film 200, moves tip 1610 so that film top surface 208 is pressed into contact with the target tissue surface 302. Wetting of the tissue to film 200 allows the physician to then withdraw applicator tip 1610, allowing the adhesion force between newly-introduced film top surface 208 and tissue 302 to overcome forces holding film 200 within tip 1610 so that film 200 slides out of a distal end 1620 of tip 1610. The physician or other operator may then use tip 1610 to push any non-adhering portions of film 200 into contact with tissue 302 before withdrawing tip 1610 to complete the procedure. A configuration of applicator 1600 having tip 1610 with an optional (integral) handle 1630 may be used in the same basic manner as described above.

Applicator 1600 is particularly suited to a disposable kit configuration, in which applicator tip 1610 (either configuration with or without handle 1630) may be preassembled with film 200 incorporated therein and provided in a sterile package for single-time use. If additional films 200 are needed, additional applicator tips 1610 with a preloaded film 200 may be used as necessary.

FIG. 22D depicts other configurations that may be suitable for use as applicator tip 1610; each of these configurations may include the features described above with respect to the embodiment of tip 1610 shown in FIGS. 22A-22C.

For the passive applicator embodiment 1600 described herein, and in particular for the embodiment of tip 1610, as with the other applicator embodiments disclosed, film 200 may be configured such that a therapeutic substance is disposed on the underside of the film underside 206, while one or more other substances (such as fibrinogen) may be disposed on the opposite, or top side 208, of film 200.

Retracting Hooks Film Delivery System

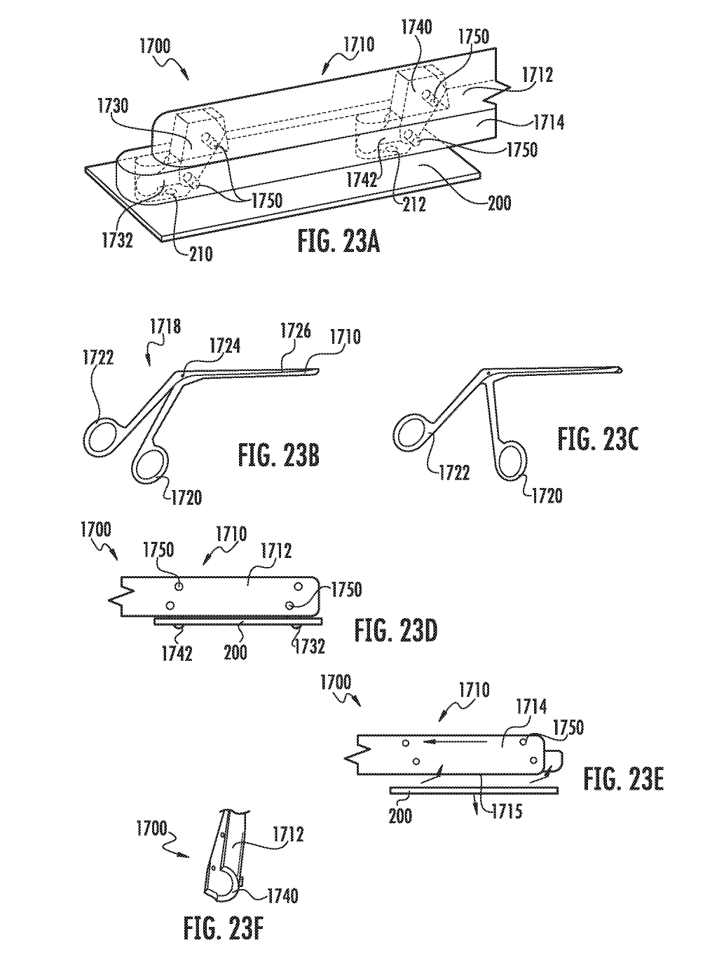

FIGS. 23A-23F depict another film delivery system in the form of a retracting hooks applicator 1700. Applicator 1700 can include a two-piece tool 1710 having upper portion 1712 and lower portion 1714. In the region of tool proximal portion 1716, handle 1718 may be included for manipulation by a physician or other operator, such that when handle first portion 1720 and handle second portion 1722 are brought closer together (FIGS. 23A, 23B), upper portion 1712 and lower portion 1714 slide relative to one another to release film 200 as described below. Alternatively, tool 1710 may be configured such that handle first portion 1720 and handle second portion 1722 may be moved apart relative to one another from the position shown in FIG. 23B to effect the relative motion to release film 200.

Tool upper portion 1712 and lower portion 1714 may be connected at various locations, such as at handle juncture 1724 and/or at tool distal portion 1726.

In the vicinity of tool distal portion 1726, at least one pivot may be included. In the embodiment of FIGS. 23A-23F, first and second pivots 1730, 1740 connect tool upper portion 1712 and tool distal portion 1714 via a set of dowel pins 1750, which can rotate on their axes within the tool, allowing pivots 1730 and 1732 to move as tool upper portion 1712 and lower portion 1714 move relative to one another. First and second hooks 1732, 1742 extend from each of pivots 1730 and 1740, respectively, and extend through apertures disposed on bottom surface 1715 of tool lower portion 1714. In turn, hooks 1732, 1742 may extend through appropriately-sized apertures 210, 212 in film 200 to secure film against tool lower portion bottom surface 1715.

During a method of use, a physician or other operator navigates applicator 1700, under optional visualization, with film 200 loaded thereon, to the body tissue site of interest 300. When it is desired to release film 200 from applicator 1700 to the tissue target surface 302, the physician or other operator squeezes handle first portion 1720 relative to handle second portion 1722 (or, in an alternative configuration, when handle first portion 1720 and handle second portion 1722 are moved apart relative to one another). Relative motion between upper and lower portions 1712, 1714 causes pivots 1730 and 1740 to move within tool lower portion 1714 via rotation of dowel pins 1750. This motion releases film 200 as it is leveraged against tool lower portion bottom surface 1715 by motion of pivots 1730, 1740. The physician or other operator may use applicator 1700, such as tool bottom portion bottom surface 1715 (which now has a flat profile given that hooks 1732 and 1742 have retracted), to press film 200 into position.

An optional support flange 1740 may be included in the location shown as a dotted line in FIG. 23F to better support film 200 during entry into the patient's body (e.g., sinus cavity). Flange may be made as an integral part of tool 1710, part of first pivot 1730, or added as a separate component. As such, flange 1740 may be made from a plastic (e.g., PET or Mylar, etc.) or a steel (e.g., stainless steel) or other metallic material. Tool and/or first and second pivots may likewise may be made from an appropriate plastic or metallic material, such as stainless steel, injection molded plastic, and the like.

Applicator 1700 is well-suited to being either a durable or disposable applicator. Applicator 1700 is amenable for the deployment of multiple films given the ease with which a film may be loaded thereon.

For the retracting hooks applicator embodiment 1700 described herein, as with the other applicator embodiments disclosed, film 200 may be configured such that a therapeutic substance is disposed on the side of the film facing tool lower portion bottom surface 1715, while one or more other substances (such as fibrinogen) may be disposed on the opposite side of the film.

Cartridge Tip Film Delivery System

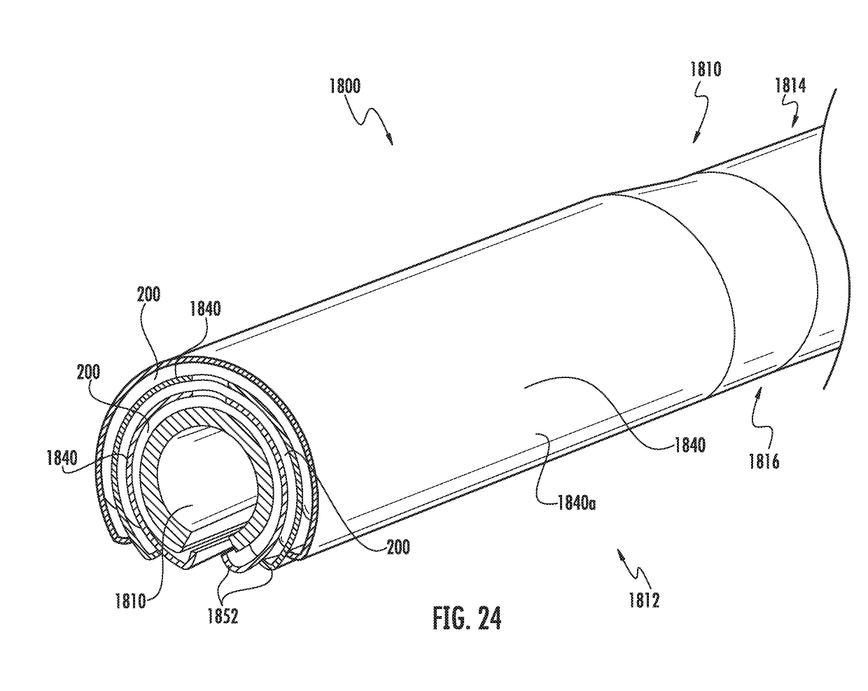

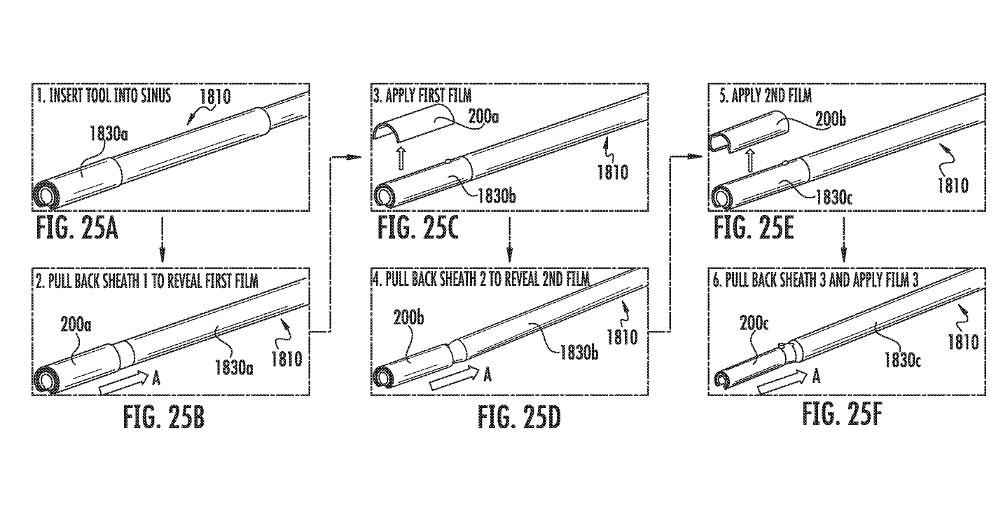

FIGS. 24-25F depict another film delivery system in the form of a cartridge tip applicator 1800. Applicator 1800 is particularly amenable to deploying multiple films as described below, although it may be used for the delivery of a single film.

As shown in the partial perspective view of FIG. 24, applicator 1800 may include tool 1810 onto which one or more films 200 are loaded on tool distal portion 1812 and that may be connected at a tool proximal end to a handle (not shown) having a retraction mechanism (not shown) suitable for revealing and deploying films 200 in a sequential nature as described below.

Tool 1810 may have an outer diameter of between about 3.0 mm and about 5.0 mm, between about 3.5 mm and 4.5 mm, about 4.0 mm, or any other diameter that is amenable to (a) carrying multiple films that are interlaid or alternatively stacked with one or more sheaths 1830 on tool distal portion 1812 and (b) tool access to and use at the location in the body where target tissue 302 resides. As tool distal portion 1812 accepts multiple layers of film and sheath thereon, it will have a larger diameter than that of tool in tool proximal portion 1814 where the film layers are not present. This results in the presence of a transition or flared section 1816 of decreasing diameter is present in the region proximal to the most proximal portion of films 200, transitioning down to a relatively constant diameter for the remainder of tool 1810.

Each sheath 1830 is disposed over tool shaft and extends from tool distal portion 1812 in a proximal direction for a length sufficient to allow retraction by a physician or other operator via any suitable technique, such as, e.g., by gripping with forceps or by use of a retraction mechanism associated with a handle. The sheath is designed such that its material properties afford close conformance to tool shaft 1830, tool distal portion 1812 in the vicinity of films 200, and in transition section 1816. Example materials from which sheath 1830 may be made includes a thin, clear extrusion of a medical grade thermoplastic material such as polyethylene terephthalate (PET).

Each sheath may have one or more rails 1852, as best seen in FIG. 24, so to facilitate gripping the edge of tool 1810 to protect film 200. Film 200 that is immediately adjacent an outer surface of tool 1810 at tool distal portion 1812 may be disposed in a recess feature on tool 1810 (not shown) to stabilize that layer of film as well as the sheaths and layers of film above it. One or more of the layers of film 200 may alternatively include a feature on its proximal end (not shown) to prevent the sheath above it from retracting that layer of film when a sheath above it is retracted in a proximal direction.

Tool 1810 may likewise may be made from an appropriate plastic or metallic material, such as stainless steel, injection molded plastic, and the like.

In a method of use, and as shown in FIGS. 25A-25F, a physician or operator may access the target tissue region 300 (such as, e.g., the sinus), under optional visualization, with tool 1810 having the desired number of film layers and sheaths incorporated therein at distal portion 1812 (FIG. 25A). Once the physician or operator is satisfied with the location of tool 1810 near the body tissue 300 of interest, he or she may proximally retract outermost sheath 1830a in the direction of arrow A in FIG. 25B by any suitable technique, exposing the outermost film 200a. Tool 1810 may then be moved such that outermost film 200a is moved into contact with the target tissue surface 302 and manipulated by, e.g., rotation and pressure, to allow film 200a to adhere to tissue surface 302 and move away from tool 1810 (FIG. 25C).

The physician or other operator will next move the tool distal portion 1812 to the desired location in the body tissue of interest 300, perhaps in a location adjacent where film 200a has been placed (or on the same site if for some reason the initial deployment was unsuccessful or film 200a was damaged during deployment). The same step is repeated with adjacent sheath 1830b, which was exposed after film 200a deployment. As sheath 1830b is proximally retracted, second film 200b is exposed to the body tissue 300 of interest and may be deployed onto a target tissue site 302 as desired (FIGS. 25D-25E). If a third film 200c is to be deployed, after positioning tool 1810 to the desired location, the physician or operator proximally retracts third sheath 1830c as with first and second sheaths, exposing third film 200c for placement on the target tissue site 302 of interest (FIG. 25F). This process may be repeated so that the desired number of films are deployed at the target tissue site 302, or multiple target tissue sites, potentially but not necessarily exhausting the supply of films loaded onto tool distal end 1812.

After the desired number of films has been deployed, the physician or other operator may withdraw tool 1810, thus completing the procedure or method of use.

Tool may be made of any suitable plastic or metallic material, such as, e.g., injection molded plastic that could be made clear so to aid in visualization of the films and sheaths.

For the cartridge tip applicator embodiment 1800 described herein, as with the other applicator embodiments disclosed, each film 200 may be configured such that a therapeutic substance is disposed on the side of the film facing the sheath 1830 immediately adjacent to and above it (e.g., the particular layer of sheath 1830 that is retracted to reveal that layer of film), while one or more other substances (such as fibrinogen) may be disposed on the opposite side of that layer of film.

Lead Screw Film Delivery System

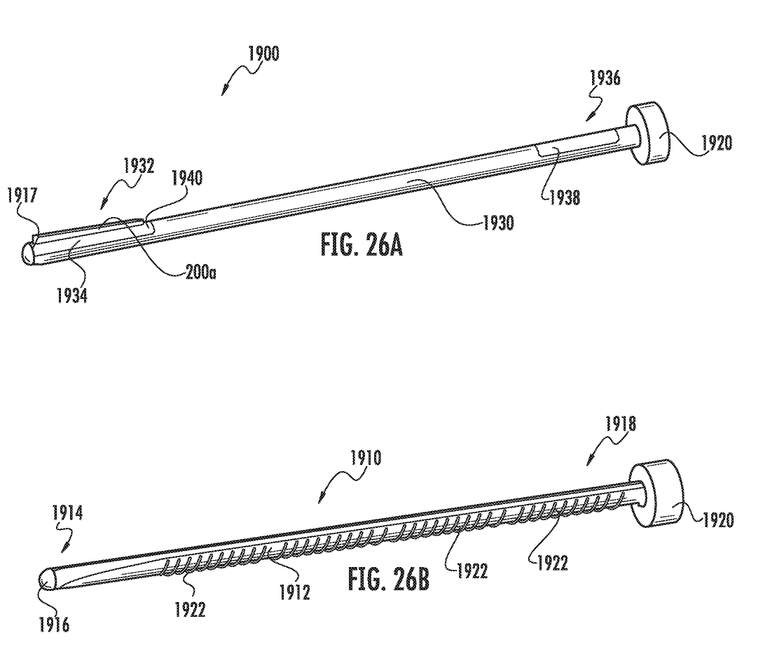

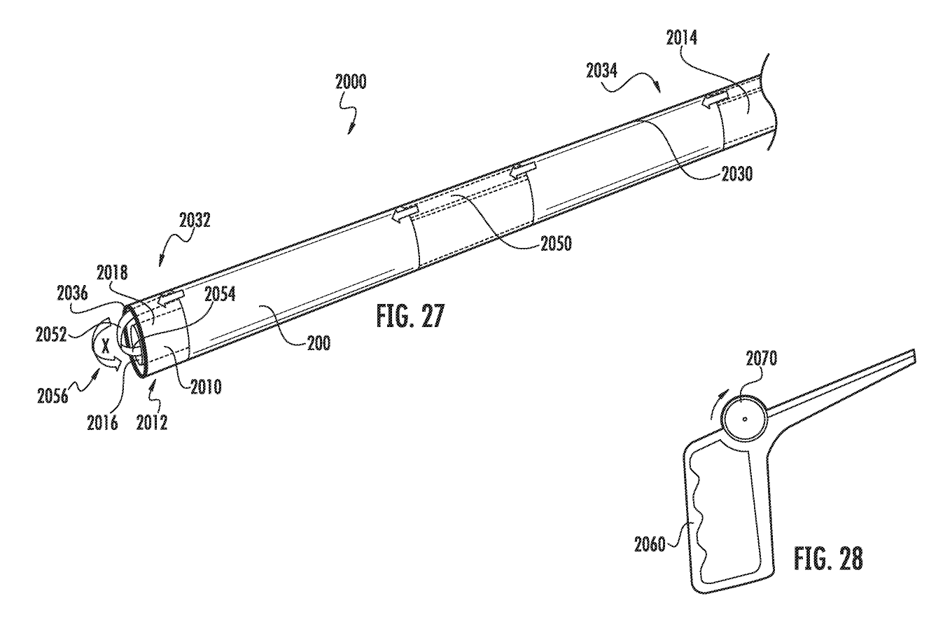

FIGS. 26A-26B depict another film delivery system of the present technology in the form of a lead screw applicator 1900. Applicator 1900 is well-suited to the controllable release of multiple films at a tissue site of interest 300.

Applicator 1900 may include lead screw 1910 over which is concentrically disposed outer tube 1930. Applicator 1900 is designed to advance one or more films 200 along a surface of lead screw 1910 as it rotates within outer tube 1930. The one or more films 200 may exit outer tube 1930 as described below to be deployed to the target tissue site 302.

Lead screw or shaft 1910 may be a plastic, metallic or composite component having an outer surface 1912, distal end 1914 with atraumatic distal tip 1916, a proximal end 1918 at which is disposed rotation handle 1920, and thread 1922 along its length on or integral with outer surface 1912. Distal tip 1916 may include an optional flange feature 1917 that can act as a stop or limiter to prevent distal movement of a film once the film reaches the distal window 1934 for deployment. The dimensions of the various features of lead screw 1910 and its accompanying thread 1922 (e.g., thread pitch, thread angle, helix angle, pitch diameter, major and minor diameters, pitch feature variations along length of screw 1910, etc.) may be appropriate for the purpose of readily and safely without damage moving one or more films 200 distally to deploy them at a body tissue site of interest. As such, shaft outer surface 1912 and/or the surfaces of part or all of thread 1922 (e.g., at the crest thread or over entire surface 1912) may be coated with an appropriate biocompatible material (e.g., PTFE or similar low-friction coating) to facilitate smooth motion of film 200 along lead screw 1910 and to prevent the film's underside of film 200 from being damaged or stuck.

Outer tube 1930 may be a tube of plastic or metallic construction having distal end 1932 with a distal opening or window 1934, proximal end 1936 with a proximal opening or window 1938, and central lumen 1940 having a diameter sized to securely accommodate lead screw 1910 therein along with one or more films while allowing for the movement of the one or more films in a distal direction along lead screw or shaft surface 1912 by rotation of handle 1920 without damage or disfigurement to the film and/or any of its contents (e.g., therapeutic substances, fibrinogen, etc.). Both distal and proximal openings or windows 1934 and 1938 are of a large enough radius angle so to allow for the optimal placement or insertion of film 200 (in the case of proximal opening 1938) and optimal exiting of film 200 (in the case of distal window 1934), and may not have the same radius angles. Windows 1924 and 1928 are generally of a length commensurate with or slightly larger than that of film 200.

During a method of use, a physician, other user or person will load one or more films onto the outer surface of lead screw 1910 by placing it in the outer tube proximal opening or window 1928 and rotating handle 1920 in the direction to advance film onto threads 1922 and in a distal direction towards shaft distal end 1914. Applicator 1900 may also be preloaded with one or more films by the manufacturer and packaged as a kit.

Next, under optional visualization, he or she guides applicator 1900 to the body tissue site of interest 300. When lead screw distal end 1914 is placed the desired location near the target tissue 302, physician or operator turns lead screw 1910 to deploy a first film by rotating handle 1920 in the appropriate direction. Rotation of lead screw 1910 may be accomplished any number of other ways, via, e.g., a motorized mechanism and/or component or components other than handle 1920. As first film 200a exits distal window 1934 via shaft 1910 rotation, the physician or operator will place film against the tissue target site 302 and may use the distal end 1914 of applicator 1900 to help first film 200a adhere to site 302 and/or to reposition film as may be desired.

If additional films are present in applicator 1900, the physician or operator will then move the applicator 1900 to the appropriate tissue location (or keep it at the same location if first film 200a did not deploy properly or was damaged, etc.) and rotate screw 1910 in the same manner to deploy and place second film 200b (not shown) at the desired location. This method may be repeated as necessary to deploy the desired number of additional films at a site 302. If a physician or other user desires to deploy a larger number of films at site 302 than are initially present or that have been loaded into proximal opening 1928, applicator 1900 may be withdrawn from the body to load one or more additional films, or another applicator 1900 with additional films may be used.

For the lead screw applicator embodiment 1900 described herein, as with the other applicator embodiments disclosed, film 200 may be configured such that a therapeutic substance is disposed on the side of the film that will be immediately adjacent the tissue at target site 302, while one or more other substances (such as fibrinogen) may be disposed on the opposite side of the film.

Belt-Guided Film Delivery System

FIGS. 27-28 depict another film delivery system in the form of a belt-guided applicator 2000. Applicator 2000 is well-suited for the delivery of multiple films as described below.

Applicator 2000 may include inner tube 2010 having distal end 2012 and proximal end 2014, outer tube 2030 having distal end 2032 and proximal end 2034, belt or string 2050 onto which is adhered one or more films 200, disposed between inner tube 2010 and outer tube 2030, and handle 2060 with an integrated or attached rotating member 2070 for advancing belt 2050.

Inner tube 2010 may be of a plastic or metallic construction and includes central lumen 2016 and an optional groove or guide (not shown) disposed on inner tube outer surface 2018 along its length along or within which a belt or string 2050 may be disposed.

Belt or string 2050 may be of any biocompatible material that is has an adhesive disposed on its outer side 2052 to facilitate adherence of film 200 thereon. Belt 2050 may be in the shape of a string, e.g., generally circular in cross section, or it may be in the shape of a ribbon, e.g., generally rectangular in cross section with the wider dimension located on outer side 2052 and generally conforming to the width of film 200. Belt 2050 may be disposed in a loop in applicator 2000 such that (a) its inner side 2054 is proximal to inner tube outer surface 2018, optionally within or along an inner tube groove or guide (not shown), and (b) it is disposed within inner tube central lumen 2016. At inner tube distal end 2012, belt 2050 takes on a distal turn 2056 as it loops from inner tube outer surface 2018 into inner tube central lumen 2016. Applicator 2000 may have one or more films pre-loaded therein or one or more films may be loaded into applicator by a physician or other person prior to or during a film deployment procedure.

Outer tube 2030 may be of a plastic or metallic construction, but may be visually opaque or transparent to facilitate viewing of belt 2050 and inner tube 2010 through outer tube central lumen 2036. Outer tube may be disposed concentrically around the inner tube 2010 such that belt 2050 is looped within outer tube 2030 and partially within inner tube 2010 as described above and shown in FIG. 27. Outer tuber 2030 serves to protect belt 2050 and its one or more adhered films 200 as applicator is disposed within a body lumen and advanced to the body tissue site or location of interest 300 for the deployment of one or more films.

Both inner tube 2010 and outer tube 2030 are open on their respective distal ends 2012, 2032, such that belt 2050 is exposed as shown in FIG. 27. The diameters of inner tube 2010 and outer tube 2030 may be chosen so to allow applicator 2000 to access body lumens and tissue 300 of interest, but to also provide the appropriate radius of curvature for belt 2050 as it exits outer tube 2030 at its distal end 2032 and enters the inner tube central lumen 2016 at its distal end 2012. This radius of curvature may be chosen to optimize reliable and consistent film deployment at distal ends 2012, 2032, depending on factors such as belt width, type of and amount of adhesive present on belt outer side 2052, the type of and amount of therapeutic material present on film 200 on the side adhering to belt outer side 2052 (if any), the type of body tissue into which film 200 is to be deployed, among other factors.

Handle 2060 may be of any configuration suitable for use by a physician or other operator, and may take on the embodiment shown in FIG. 28. Integrated with or disposed on or within handle 2060 is rotating member 2070 having outer surface 2072 around which is disposed belt 2050. In the embodiment of FIG. 28, rotating member 2070 is designed for manual operation, such as via a physician or other operator's thumb, in the direction X shown. Such rotation advances belt 2050 in a continuous loop through outer tube central lumen 2036 and into inner tube central lumen 2016 as described above. One or more films may be added as needed onto belt outer side 2052 in the vicinity of handle 2060 and rotating member 2070 prior to a position where belt 2050 moves into outer tube central lumen 2036. Any number of other configurations for rotating member 2070 may be used, including those that may be operated automatically (e.g., battery or electric-powered) provided the belt 2050 may be advanced in a controlled manner suitable for the deployment of films 200 therefrom.

In a method of use, applicator 2000 may come pre-loaded with one or more films 200 on belt 2050 or a physician, other user, or other person may load one or more films 200 onto belt 2050 as needed prior to the film deployment procedure. The physician or other user, under optional visualization, will guide applicator 2000 to the desired body tissue location 300 for the deployment of one or more films 200. As the distal end of inner tube 2012 and outer tube 2032 are in the vicinity of the tissue target surface 302, the physician or other user rotates rotating member 2070 to advance belt 2050 such that first film 200a that is adhered thereto moves out of applicator 2000 in the vicinity of belt distal turn 2056. As first film 200a encounters distal turn 2056, the belt's radius of curvature aids to detach first film 200a from belt outer side 2052, allowing it to be deployed on tissue target site 302. Any component of applicator 2000 may be used after first film 200a's deployment to aid in the film's positioning or repositioning on tissue target site 302. If desired, the physician or other operator further rotates rotating member 2070 to advance a second film 200b out of applicator 2000 and deploy second film 200b as described above. This process may be repeated as necessary to deploy additional films 200.

For the belt-guided applicator embodiment 2000 described herein, as with the other applicator embodiments disclosed, film 200 may be configured such that a therapeutic substance is disposed on the side of the film facing belt 1450, while one or more other substances (such as fibrinogen) may be disposed on the opposite side of the film.

Push Tube Film Delivery System

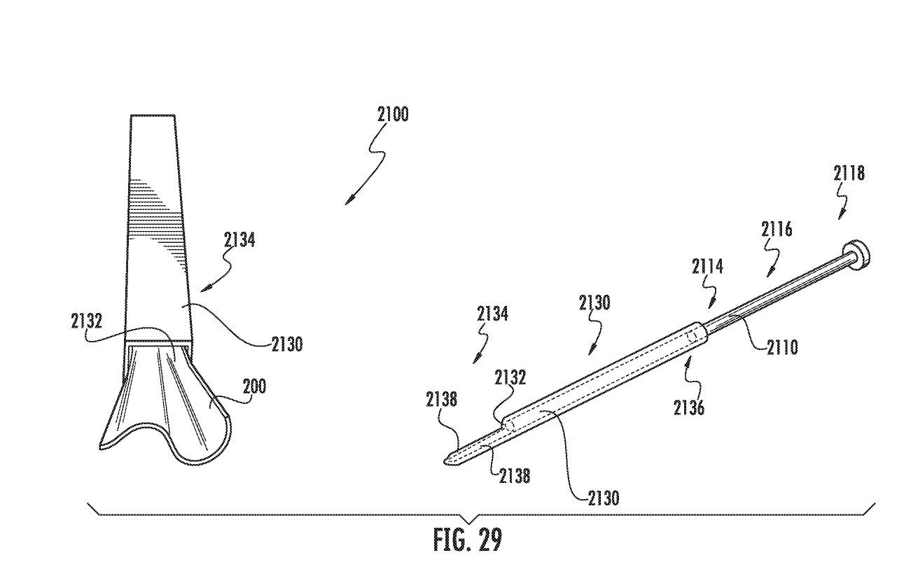

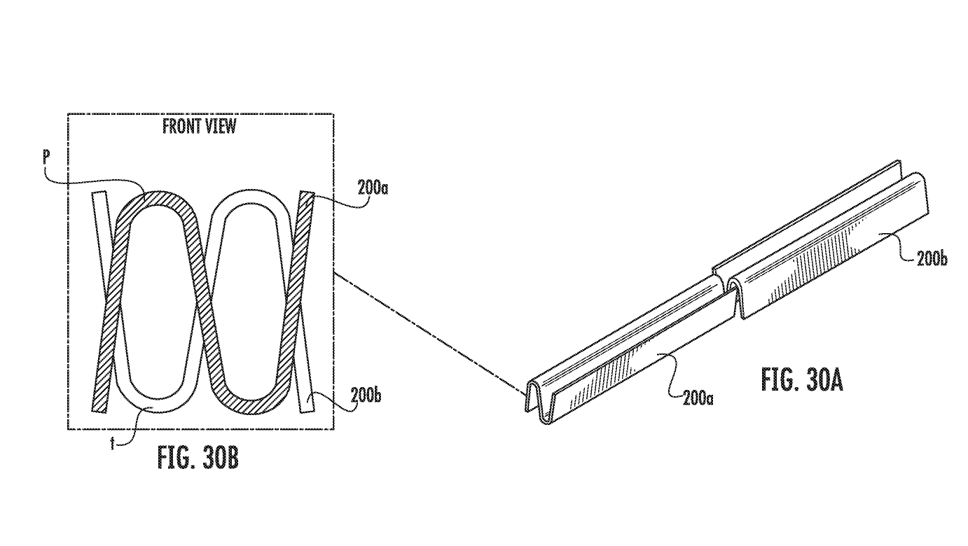

FIGS. 29-30B depict another film delivery system in the form of push tube applicator 2100. Applicator 2100 is well-suited for the delivery of multiple films as described below.

Applicator 2100 may include pusher 2110 and outer tube 2130. Pusher 2110 is configured to be disposed within outer tube 2130 to effect the ejection of one or more films 200 out of outer tube 2130 as described below.

Pusher 2110 may be made of any suitable plastic or metallic material (e.g., molded plastic or stainless steel) and may include an open area 2012 at pusher distal end 2114 and plunger element 2116 on pusher proximal end 2118. Closed region 2120 may be located proximal to open area 2012 into which one or more films may be loaded; closed region 2120 serves to protect one or more films prior to their deployment to the body tissue site of interest 300.

Outer tube 2130 may be made from any suitable plastic or metallic material (such as, e.g., a plastic extrusion or stainless steel). In FIG. 29, outer tube 2130 may include central lumen 2132 that takes on, alternatively, generally square and circular cross-sectional shapes, although other cross-sectional shapes, including for example rectangular or elliptical, are contemplated. The cross-sectional area of outer tube 2130 may be sized to accommodate the one or more folded films 200 generally to match the shape of folded films as they are disposed within central lumen 2132. An outer tube distal end 2134 is open so to allow the deployment or ejection of one or more films therethrough. An outer tube proximal end 2136 is open and configured to accept pusher 2110 therethrough. One or more ridges, or rails 2138 may be disposed as shown in FIG. 29 on outer tube 2130 to securely carry one or more films 200. As shown, ridges 2138 are configured as part of outer tube 2130 such that the one or more films carried thereon generally take on an “N” shape, or “sine wave” curve, as they are disposed on rails 2138 within outer tube central lumen 2132. This shape helps to protect the one or more films from overlapping, or stacking onto one another, as it is generally desirable to have the films, if there are more than one, be configured in a serial non-overlapping fashion on rail 2138. As shown in FIG. 30B, if more than one film is present, immediately adjacent films 200a, 200b may be folded such that they have an alternating or mirror image periodicity in which a peak p of film 200a matches a trough t of film 200b. This pattern, which may be repeated as multiple films are used, also serves to prevent films 200a and 200b from overlapping and ensure an orderly deployment as described below. Other configurations for such adjacent films, including non-mirror image profiles or combinations of mirror image and non-mirror image profiles, are contemplated.

In a method of use, applicator 2100 may come pre-loaded with one or more films 200 or a physician, other user, or other person may load one or more films 200 into applicator 2100, as needed, prior to the film deployment procedure. The physician or other user, under optional visualization, will guide applicator 2100 to the desired body tissue location 300 for the deployment of one or more films 200. As outer tube distal end 2134 is moved into the vicinity of the tissue target surface 302, the physician or other user advances plunger element 2116 in a distal direction to advance pusher 2110 through outer tube central lumen 2132, pushing one or more films 200 along rails 2138. As pusher 2110 continues to advance, first film 200a moves out of applicator 2100; specifically, out of outer tube central lumen 2132, at outer tube distal end 2314. First film 200a may be made of a material having sufficient elasticity to unfold into a generally flat shape upon deployment from applicator 2100, or it may still take on the generally curved shape that it had while inside applicator 2100. At this point, the physician or other operator may use the applicator 2100 to manipulate first film 200a onto target tissue surface 302a and to ensure first film 200a conforms thereto. Any component of applicator 2100 may be used after first film 200a deployment to aid in the film's positioning or repositioning on tissue target site 302. If desired, the physician or other operator further advances plunger element 2116 in a distal direction to advance second film 200b out of applicator 2100; specifically, out of outer tube central lumen 2132, and deploy second film 200b as described above. This process may be repeated as necessary to deploy any or all remaining additional films 200.

For the push tube applicator embodiment 2100 described herein, as with the other applicator embodiments disclosed, film 200 may be configured such that a therapeutic substance is disposed on one side of the film while one or more other substances (such as fibrinogen) may be disposed on the opposite side of the film.

Although the technology has been described in terms of particular embodiments and applications, one of ordinary skill in the art, in light of this teaching, can generate additional embodiments and modifications without departing from the spirit of or exceeding the scope of the exampled technology. Accordingly, it is to be understood that the drawings and descriptions herein are proffered by way of example to facilitate comprehension of the technology and should not be construed to limit the scope thereof.

What is claimed:

1. A delivery apparatus for delivering a biocompatible film to a nasal cavity or a sinus cavity of a patient, the delivery apparatus comprising:
a sheath comprising a distal end, a proximal end, and a lumen extending therebetween, the distal end of the sheath sized and shaped for insertion through a nostril of the patient and for advancement to at least one of the nasal cavities or the sinus cavity;
a handle disposed adjacent to the proximal end of the sheath, the handle comprising an actuator;
a shaft coupled to the actuator and disposed within the lumen of the sheath; and
a curved end effector coupled to a distal end of the shaft and comprising a convex outer surface configured to have the biocompatible film disposed thereon during delivery, the curved end effector configured to transition from a delivery state, wherein the curved end effector is disposed within the lumen of the sheath, to a deployed state, wherein the curved end effector is exposed past the distal end of the sheath to deliver the biocompatible film to tissue at the nasal cavity or the sinus cavity, responsive to actuation at the actuator.

2. The system of claim 1, wherein the shaft comprises a groove extending between a proximal end and a distal end of the shaft, and wherein the sheath comprises a folded portion sized and shaped to align with the groove of the shaft.

3. The system of claim 2, wherein the folded portion of the sheath comprises a slit configured to provide strain relief as the shaft is disposed within the lumen of the sheath.

4. The system of claim 1, wherein a longitudinal axis of a distal end of the shaft is offset by a predetermined angle to a longitudinal axis of a proximal end of the shaft.

5. The system of claim 1, wherein the shaft comprises a channel disposed along an upper portion of the shaft and configured to receive an actuator post.

6. The system of claim 1, wherein the curved end effector provides a uniform gap between an inner surface of the sheath and the biocompatible film when the sheath is disposed over the convex outer surface of the curved end effector.

7. The system of claim 1, wherein the curved end effector comprises a flared distal surface.

8. The system of claim 7, wherein the convex outer surface of the curved end effector has a radius of curvature decreasing axially in a direction from the flared distal surface toward the proximal end.

9. The system of claim 7, wherein the convex outer surface of the curved end effector has a constant radius of curvature along a longitudinal axis of the curved end effector.

10. The system of claim 7, wherein the flared distal end comprises an atraumatic tip configured to minimize trauma or damage to the tissue during delivery.

11. The system of claim 7, wherein the curved end effector comprises left and right rails extending from the flared distal surface longitudinally along at least a portion of opposite lateral ends of the curved end effector.

12. The system of claim 11, wherein the left and right rails are configured to facilitate consistent and reliable axial sliding of the shaft within the sheath.

13. The system of claim 11, wherein the left and right rails are configured to keep fluid out of a vicinity of the biocompatible film to avoid premature wetting thereof.

14. The system of claim 1, wherein the convex outer surface of the curved end effector comprises at least one indented portion extending longitudinally along the convex outer surface, the at least one indented portion having a radius of curvature less than a radius of curvature of the curved end effector, such that a surface of the indented portion is separated a predetermined distance from the biocompatible film when the biocompatible film is disposed on the convex outer surface of the curved end effector.

15. A method for delivering a biocompatible film to a nasal cavity or a sinus cavity of a patient, the method comprising:
inserting a distal end of a sheath through a nostril of the patient, the sheath having a shaft disposed within a lumen of the sheath, the shaft having a curved end effector coupled to a distal end of the shaft and comprising a convex outer surface configured to have the biocompatible film disposed thereon during delivery;
advancing the distal end of the sheath to at least one of the nasal cavities or the sinus cavity;
actuating an actuator disposed adjacent a proximal end of the sheath to transition the curved end effector from a delivery state, wherein the curved end effector is disposed within the lumen of the sheath, to a deployed state, wherein the curved end effector is exposed past the distal end of the sheath; and
delivering the biocompatible film to tissue at the nasal cavity or the sinus cavity.

16. The method of claim 15, wherein a longitudinal axis of a distal end of the shaft is offset by a predetermined angle to a longitudinal axis of a proximal end of the shaft.

17. The method of claim 15, wherein the curved end effector provides a uniform gap between an inner surface of the sheath and the biocompatible film when the sheath is disposed over the convex outer surface of the flared end effector.

18. The method of claim 15, wherein the curved end effector comprises a flared distal surface.

19. The method of claim 18, wherein the curved end effector comprises left and right rails extending from the flared distal surface longitudinally along at least a portion of opposite lateral ends of the curved end effector, the left and right rails configured to facilitate consistent and reliable axial sliding of the shaft within the sheath.

20. The method of claim 15, wherein the convex outer surface of the curved end effector comprises at least one indented portion extending longitudinally along the convex outer surface, the indented portion having a radius of curvature less than a radius of curvature of the curved end effector, such that a surface of the indented portion is separated a predetermined distance from the biocompatible film when the biocompatible film is disposed on the convex outer surface of the curved end effector.
